(19) United States
(12) Patent Application Publication
(10) Pub. No.: US 2006/0275764 A1
(43) Pub. Date: Dec. 7, 2006

(54) Method for determining predisposition to manifestation of immune system related diseases

(75) Inventors: Steffen Thiel, Risskov (DK); Jens Christian Jensenius, Odense M (DK); Lars Fugger, Headington (GB); Kristian Stengaard-Pedersen, Risskov (DK)

Correspondence Address: BROWDY AND NEIMARK, P.L.L.C.;624 NINTH STREET, NW, SUITE 300, WASHINGTON, DC, 20001-5303, US

(73) Assignees: Aarhus Universitet, Aarhus (DK); Aarhus Amt, Hoejbjerg (DK)

(21) Appl. No.: 10/537,507

(22) PCT Filed: Dec. 2, 2003
(86) PCT No.: PCT/DK03/00827

§ 371 Date: Jun. 3, 2005

(30) Foreign Application Priority Data
Dec. 3, 2002 (PA) PA 2002 01867
Dec. 9, 2002 (PA) PA 2002 01892
May 15, 2003 (PA) PA 2003 00742

(51) Int. Cl. C12Q 1/68; C07H 21/04; A61K 38/46; A61K 48/00; C12P 21/06; C12N 9/64; C07K 16/40

(52) U.S. Cl. 435/6; 435/69.1; 435/226; 435/320.1; 435/325; 536/23.2; 530/388.26; 514/44

(57) ABSTRACT

The present invention relates to methods for determining predisposition to a manifestation of immune system related diseases, in particular infections, associated with a mutation in the human MASP-2 gene. The invention also features oligonucleotides, polypeptides, peptide fragments and antibodies which are used in the above methods as well as for the manufacture of a medicament for treatment of a disease associated with pathological activity of the lectin-complement pathway. Moreover, the invention provides a series of gene therapy vectors and a kit for diagnosis of the disease associated with a mutation in the human MASP-2 gene.

### FIELD OF INVENTION

The present invention relates to methods for determining predisposition to a manifestation of immune system related diseases, in particular infections, associated with a mutation in the human MASP-2 gene. The invention also features oligonucleotides, polypeptides, peptide fragments and antibodies which are used in the above methods as well as for the manufacture of a medicament for treatment of a disease associated with pathological activity of the lectin-complement pathway. Moreover, the invention provides a series of gene therapy vectors and a kit for diagnosis of the disease associated with a mutation in the human MASP-2 gene.

### BACKGROUND OF INVENTION

The innate immune defence systems provide essential protection during the initial phases of an infection. A number of cellular and humoral components participate in destroying microorganisms, which have penetrated trough the mechanical barriers of the body. The clinical significance of the immune defence is reflected in the large number of different immunodeficiencies identified, many of which being genetically determined.

Mannan-binding lectin (MBL) is an important constituent of the innate immune system. This protein binds via multiple lectin domains to the repeating sugar arrays that decorate microbial surfaces, and become able to activate the complement system of blood through activation of specific proteases called MBL-associated serine proteases (MASPs). To be capable to activate the complement the MBL-MASP complexes should consist of at least one of three MASPs: MASP-1, MASP-2 and/or MASP-3, and a small protein, MAp-19, which is generated by alternative splicing of the MASP-2 gene, and does not possess the enzymatic activity.

MASP-2 cleaves the complement factors C4 and C2 generating the C3 convertase, C4bC2b, activating thereby the lectin pathway. Activation of C3 via MASP-2 additionally recruits the alternative pathway and initiates the formation of the membrane attack complex (MAC). The deposited fragments of complement factors are ligands for receptors on phagocytic cells and thus act as important opsonins while other fragments promote inflammation.

Structurally the MASP-2 polypeptide is composed of an N-terminal CUB1 domain, followed by an epidermal growth factor (EGF) domain, a second CUB domain (CUB 2), two complement control domains (CCP1 and CCP2), and serine protease domain. The domains of MASP-2 are involved in functional interactions with different proteins of the complement system. The CUB1 and EGF domains have been suggested play a major role in association of MASP-2 with MBL in the MBL-MASP complexes.

Recently, it has been shown that other serum lectins, L- and H-ficolins are also capable of activation the complement system via association with MASPs.

There is an increasing literature suggesting that deficiency of the lectin pathway of the complement system, which mainly results from three relatively common single point mutations in exon 1 of the MBL gene, predisposes both to infection by extracellular pathogens and to autoimmune disease.

### SUMMARY OF INVENTION

The present invention concerns polymorphism of the human MASP-2 gene associated with impaired defence capabilities of the innate immune system. Accordingly, one aspect the present invention relates to methods for determining a predisposition for a manifestation of immune system related diseases in an individual carrying at least one mutation in the MASP-2 gene. Thus, the invention discloses
a method for determining a predisposition for a manifestation of an immune system related disease in an individual comprising determining in a biological sample isolated from said individual the presence or absence of a polymorphism within the amino acid sequence of the MASP-2 protein as identified in SEQ ID NO: 1 and/or within the amino acid sequence of the MAp-19 protein as identified in SEQ ID NO: 2, said polymorphism being a substitution, deletion and/or addition of least one amino acid residue within said sequences, and
a method for determining a predisposition for a manifestation of immune system related diseases comprising determining the presence or absence of a polymorphism within the human MASP-2 gene, said polymorphism being a substitution, deletion or addition of at least one nucleotide within said gene.

In another aspect the invention relates to isolated oligo- and polynucleotides, among which the preferred are
an isolated polynucleotide sequence encoding the MASP-2 polypeptide (SEQ ID NO: 1) having Gly at positon 105,
an isolated polynucleotide encoding the MAp-19 polypeptide (SEQ ID NO: 2) having Gly at positon 105, and
an isolated oligonucleotide comprising at least 10 contiguous nucleotides of SEQ ID NO: 3, or the corresponding complementary strand, said oligonucleotide sequence comprising the G allele in position 359, said position corresponding to the sequence set forth in SEQ ID NO: 3, or the corresponding allele of the complementary strand.

Furthermore, the invention relates to isolated polypeptides, among which the preferred are
a polypeptide comprising the amino acid sequence set forth in SEQ ID NO: 1 or a fragment thereof, said polypeptide or said fragment comprising Gly in position corresponding to position 105 of said sequence, and
a polypeptide comprising the amino acid sequence set forth in SEQ ID NO: 2 or a fragment thereof, said polypeptide or said fragment comprising Gly in position corresponding to position 105 of said sequence.

The invention also relates to peptide fragments derived from the amino acid sequence set forth in SEQ ID NO: 1, among which
a peptide fragment having a size in a range from 5 to 160 amino acids comprising at least 5 amino acid contiguous sequence, said sequence corresponding to amino acid residues 100-105, 101-106, 102-107, 103-108, 104-109 and/or 105-110 of the sequence set forth in SEQ ID NO: 1, and
a peptide fragment having a size in a range from 5 to 160 amino acids comprising at least 5 amino acid contiguous sequence, said sequence corresponding to amino acid residues 100-105, 101-106, 102-107, 103-108, 104-109 and/or 105-110 of the sequence set forth in SEQ ID NO: 1, wherein Gly in position 105 of said sequence is substituted for Asp.

It is another important aspect of the present invention to use the above oligo- and polynucleotides, polypeptides and peptide fragments for modulation of activity of the MBL-complement pathway. The modulation of the MBL-complement pathway by the above molecules may be either activation of said pathway in an individual having a predisposition to a manifestation of immune system related diseases, or it may be inhibition of the pathway, when a high activity thereof is not desirable. Thus the invention relates to
i) use of a polypeptide comprising an amino acid sequence set forth in SEQ ID NOS: 1 or 2, or a fragment thereof, said polypeptide or said fragment comprising Gly in position 105 of said sequences, for inhibition of activity of the lectin-complement pathway;
ii) use of a peptide fragment having a size in a range from 5 to 160 amino acids comprising at least 5 amino acid contiguous sequence, said sequence corresponding to amino acid residues 100-105, 101-106, 102-107, 103-108, 104-109 or 105-110 of the sequence set forth in SEQ ID NO: 1 for inhibition of activity of the lectin-complement pathway;
iii) use of a polypeptide comprising any of the amino acid sequences set forth in SEQ ID NOS: 1 or 2, or fragments thereof, said polypeptides or said fragments comprising the glycine residue in position 105 of said sequences for the manufacture of a medicament for treatment of therapeutic conditions associated with pathologically high activity of the lectin-complement pathway;
iv) use of an oligonucleotide and/or polynucleotide as the defined above for the manufacture of a medicament for treatment of therapeutic conditions associated with pathologically high activity of the lectin-complement pathway,
v) use of a polypeptide comprising an amino acid sequence set forth in SEQ ID NO: 1 or 2, or a fragment thereof for rescue of the activity of the lectin-complement pathway in an individual having a polymorphism in the MASP-2 gene.

Moreover, the invention discloses a kit for the prediction of an increased risk developing a manifestation of an immune system disease in a subject. The kit of the invention comprises at least one probe comprising an oligonucleotide sequence as the defined above and/or at least one probe comprising an antibody as the defined below.

An antibody capable of selectively binding to a polypeptide comprising Gly in position 105 according to SEQ ID NO: 1 or SEQ ID NO: 2 and not to the polypeptide having the amino acid sequence of SEQ ID NO: 1 or SEQ ID NO: 2 is another aspect of the invention.

The invention also concerns an antibody capable of recognition of an epitope comprising Asp in position 105 corresponding to the sequence set forth in SEQ ID NO:1 or SEQ ID NO:2. This antibody may to be used for the manufacture of a medicament for inhibition of the lectin-pathway, when a high activity thereof is not desirable.

An important aspect of the invention concerns providing a series of gene therapy vectors. One gene therapy vector of the invention may be used for the treating pathologic conditions associated with a low activity of the MBL-pathway in a subject carrying the G allele of the MASP-2 gene in the position corresponding to nucleotide no. 359 of the sequence set forth in SEQ ID NO: 3 or SEQ ID NO: 4. This vector comprises a coding DNA sequence for the MASP-2 and/or MAp-19 polypeptide. Another gene therapy vector comprising the nucleotide sequence of SEQ ID NO: 3 having a polymorphism within said sequence, said polymorphism being the single nucleotide substitution A→G in position no 359 corresponding to nucleotide no. 359 of the sequence set forth in SEQ ID NO: 3 or SEQ ID NO: 4, may be used for the treating therapeutic conditions associated with a pathologically high activity of the MBL-pathway. Preferably the gene therapy vectors of the invention are operably linked to a promoter sequence capable of directing the in vivo expression of the mutated or wild type MASP-2 and/or Map-19 polypeptides.

Finally, the invention relates to a method of treatment of an individual having a predisposition to a manifestation of an immune system related disease comprising
i) identifying a polymorphism in the MASP-2 gene of said individual and
ii) administering to said individual an effective amount of a polypeptide comprising SEQ ID NO:1 and/or polypeptide comprising SEQ ID NO:2.

### DESCRIPTION OF DRAWINGS

FIG. 1 shows the analyses of the MBL pathway. MBL complexes were examined for the presence of MAp19 and MASP-2, MASP-1 or MASP-3. Plasma from one normal (control) person (●) and from the patient (◯) was diluted in hypertonic buffer and incubated in mannan coated microtiter wells, and the bound complexes analyzed with antibody reacting with MASP-2 and MAp19 (panel A), anti-MASP-1 antibody (panel B), anti-MASP-3 antibody (panel C). Bound antibody was detected with anti-Ig antibody labeled with europium. Other normal plasma were tested with results similar to the normal plasma depicted in the figure. Restoring the MBL pathway activity with rMASP-2 is shown in panel D. Culture supernatant from an rMASP-2 cell culture (●) or from a control cell culture (◯) were added to patient serum. The serum was then incubated in mannan coated wells allowing for MBL-MASP complexes to bind and for C4b to be deposited in the wells. Deposited C4 was estimated using anti-C4 antibody. Panel E illustrates the lack of effect of adding MBL to patient plasma: MBL was added to patient plasma (●) or to MBL deficient plasma (◯) and incubated in mannan coated wells. After wash C4 was added and deposition of C4b measured. The MBL concentration in the wells are given on the x axis, with regards to patient plasma the sums of endogenous and added MBL are given. Panel F: Quantification of MASP-2 and MAp19 in plasma. Plasma was incubated with anti-MAp19/MASP-2 beads, bound material was eluted and analyzed by SDS-PAGE Western blotting using anti-MAp19/MASP-2 antibody. Control plasma and the patient plasma were analyzed at several dilutions.

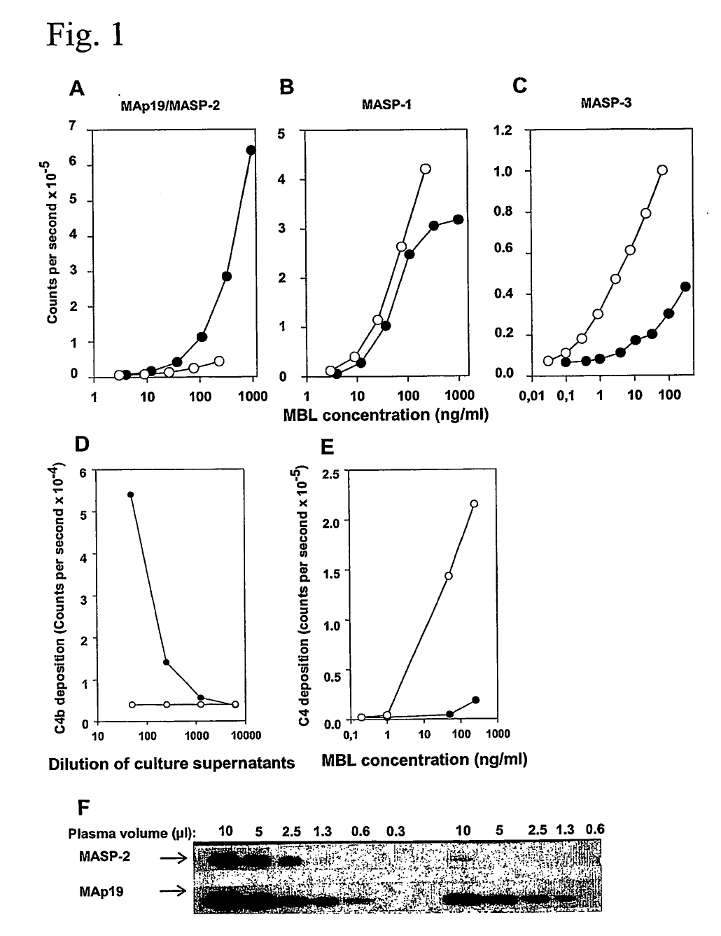

FIG. 2 depicts location of the MASP-2 mutation. Panel A, the structure of MASP-2, MAp19 and the encoding gene. The position of the mutation in the CUB1 domain is indicated (CUB indicates the CUB domains, E the EGF-like domain, CCP the complement control protein domains and SP the serine protease domain). Panel B, nucleotide and amino acid sequences flanking the mutation in CUB1 (amino acid 105 in the mature protein). Panel C, the position of the mutated amino acid shown in the crystal structure of the homologous CUB domain of sperm adhesin.²¹

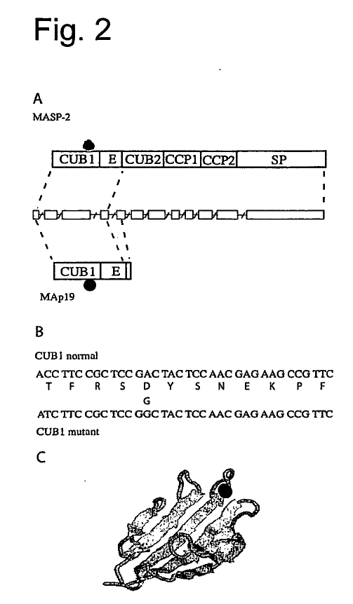

FIG. 3 shows data on MASP-2 levels in plasma samples. The levels of MASP-2 were estimated by a double antibody assay. Standard curves were constructed using a pool of normal plasma arbitrarily assigned the value of 1 unit MASP-2 per ml. The mean levels, the 10^{th} and 90^{th} percentile, and outliers are shown for the 86 homozygous (D120/D120) control individuals and for 11 heterozygous (D120/G120) control individuals together with the 5 heterozygous family members of the patent; p<10⁻⁸. Also shown is the value for the patient (G120/G120).

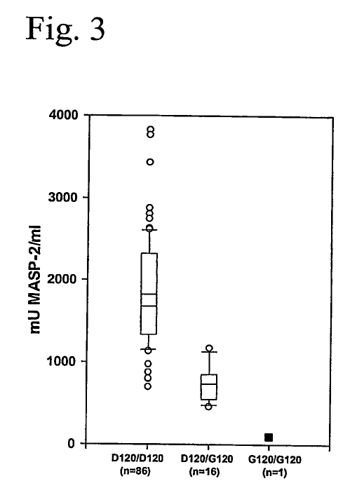

FIG. 4 represents the results of functional analysis of recombinant wild type and mutant type MASP-2. The binding of MASP-2 to MBL (100 ng/well) was detected with anti-MASP-2 antibody after application of the mixtures to anti-MBL-coated wells (panel A). Filled and open symbols indicate wild type and mutant type, respectively. The function of the MBL/MASP complexes, i.e., their C4-cleaving capacity is illustrated in panel B. Panel C represents a Western blot of the two recombinant MASPs.

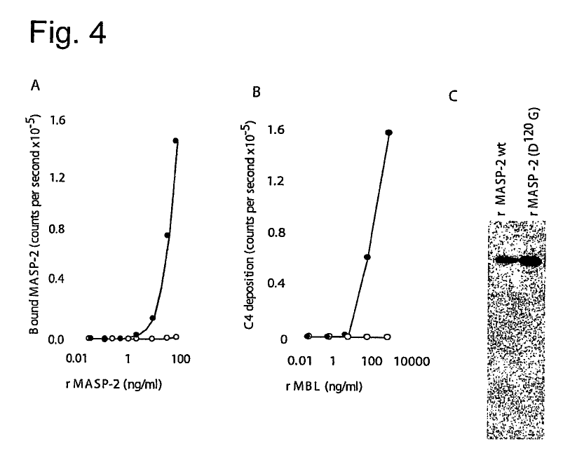

### DETAILED DESCRIPTION OF THE INVENTION

Method for Determining a Predisposition for a Manifestation of Immune System Related Diseases

It is an objective of the present invention to provide methods for determining a predisposition for a manifestation of immune system related diseases.

By the term “predisposition” in the present context is meant a conditional state, which renders the individual to be more prone to disease development compare to other individuals.

By the term “manifestation of an immune system related disease” is meant a disease or disorder resulting from the immune system disease, for example an infection.

In one aspect, the invention concerns a disease selected from the group comprising infectious diseases. An infection is a disease developed in an individual due to infection by a microorganism, parasite or virus. The invention preferably concerns pathogenic microorganisms selected from the group comprising pathogenic bacteria or fungi. Preferred embodiments for infectious species of bacteria, fungi, parasites and viruses are described below.

In another aspect the invention concerns immune system related diseases associated with the presence of a polymorphism within the amino acid sequence of the MASP-2 protein as identified in SEQ ID NO: 1 and/or within the amino acid sequence of the MAp-19 protein as identified in SEQ ID NO: 2.

By the term “polymorphism” in the present context is meant the existence of a variant(s) of the amino acid sequence of a polypeptide. By “variant” is mean a polypeptide wherein at least one amino acid is substituted, deleted and/or added to the sequence as compared to SEQ ID NO: 1 or SEQ ID NO: 2. In particular, the polymorphism may be due to a single nucleotide polymorphism (SNP) of a nucleotide sequence encoding the polypeptide.

In a preferred embodiment the invention relates to diseases, which are associated with the polymorphism of least one amino acid residue of the sequence as defined in SEQ ID NO: 1 or 2. In a more preferred embodiment the invention concerns diseases associated with the polymorphism of at least one amino acid residue located within a fragment of MASP-2 consisting of CUB1, EGF, CUB2, CCP1 and CCP2 structural domains. More preferably, if the polymorphism is located within a fragment of MASP-2 consisting of CUB1, EGF, CUB2 and CCP1 domains. It is even more preferred embodiment, if the polymorphism is located within a fragment of MASP-2 or MAp-19 consisting of CUB1 and EGF domains corresponding to 1-166 amino acid residues according to SEQ ID NO: 1 or 2. Even more preferably if the polymorphism is located between position 80 and position 120 corresponding to the sequences set forth in SEQ ID NO: 1 or 2. Still, it is further more preferred embodiment, if the polymorphism is a substitution or deletion of Asp in position 105 corresponding to the position of the amino acid sequences set forth in SEQ ID NO: 1 or 2. And, in the most preferred embodiment the invention is related to a disease associated with the substitution/mutation of Asp in position 105 in a sequence identified as SEQ ID NO: 1 or SEQ ID NO: 2 to Gly.

Thus, it is an objective of the present invention to provide a method for determining a predisposition for a manifestation of immune system related diseases in an individual carrying the above polymorphism comprising determining in a biological sample isolated from said individual the presence or absence of said polymorphism within the amino acid sequence of the MASP-2 protein as identified in SEQ ID NO: 1 and/or within the amino acid sequence of the MAp-19 protein as identified in SEQ ID NO: 2.

Another objective of the present invention is to provide a method for determining a predisposition for a manifestation of immune system related diseases comprising determining the presence or absence of a polymorphism within the MASP-2 gene, such as within the coding DNA sequence (SEQ ID NO: 3) of the human MASP-2 gene.

By the term “polymorphism” in the present context is meant the existence of variants of the oligonucleotide sequence encoding a polypeptide. By “variant” is meant a nucleotide sequence wherein at least one nucleotide is substituted as compared to SEQ ID NO: 3.

In a preferred embodiment, the above method concerns a polymorphism of the DNA sequence encoding the MASP-2 polypeptide, said polymorphism being a substitution, deletion or addition of at least one nucleotide within the coding sequence of the human MASP-2 gene. In a more preferred embodiment, the polymorphism concerns a mutation located in the sequence encoding a fragment of MASP-2 consisting of CUB1, EGF, CCP1, CUB2 and CCP2 domains. More preferably the polymorphism is located in the sequence encoding the CUB1, EGF CUB2 and CCP1 domains of MASP-2. Even more preferably the polymorphism is a mutation in of the coding sequence of the CUP1 and EGF domains of MASP-2 and/or MAp-19. It is further preferably, if the polymorphism is located in the coding sequence of the CUB1 domain of MASP-2 and/or MAp-19. The preferred position of the mutation in the CUB1 encoding sequence is between nucleotides no. 319 and 399 corresponding to the positions of SEQ ID NO: 3 or 4. In the most preferred embodiment the method relates to the polymorphism being a single nucleotide substitution/mutation A→G in position 380.

Other embodiments of the invention concern any nucleic sequences encoding the polypeptides comprising the polymorphism as the defined above.

The invention also concerns the polymorphism located in the regulatory elements of the MASP-2 gene, such as for example a promotor region.

Determination of Polymorphism

According to the above methods the predisposition of an individual to a manifestation of immune system related diseases is in one embodiment determined by determining a polymorphism within the MASP-2 and/or MAp-19 polypeptides, and/or a polymorphism within the human MASP-2 gene.

Position of Polymorphism

The invention relates in particular to any polymorphism located within the range of the exons of the human MASP-2 gene.

Preferably, the polymorphism used for detecting the predisposition is a polymorphism or mutation located in the region of the exon(s) encoding the CUB-1 domain. More preferably, said mutation or polymorphism is to be present in the nucleotide sequence set forth in SEQ ID NO: 3 or 4 or in a sequence having at least 90% sequence identity with SEQ ID NO: 3 or 4, or in a sequence being complementary to one of these sequences. SEQ ID NO: 3 represents the cDNA sequence encoding the MASP-2 protein and consists of the sequences of the exons a, c and d of the MASP-2 gene. SEQ ID NO: 4 represents the cDNA sequence encoding the MAp-19 protein and consists of the sequences of the exon a and exon b of the MASP-2 gene (Stover et al., J. Immun. 1999, 162:3481-90).

More preferably the polymorphism or mutation is located in the region encoding a fragment of the amino acid sequence of the MASP-2 protein identified as SEQ ID NO: 1 or the MAp-19 protein identified as SEQ ID NO: 2 from position 80 to 120. Even more preferably said polymorphism or mutation causes the substitution of an amino acid residue in the MASP-2 or MAp-19 protein sequences in position 105. The substitution may be a conservative amino acid substitution or a non-conservative amino acid substitution.

Conservative amino acid substitutions are considered within the following groups of amino acids. Substitutions outside the following groups are considered non-conservative amino acid substitutions.

P, A, G, S, T (neutral, weakly hydrophobic)

Q, N, E, D, B, Z (hydrophilic, acid)

H, K, R (hydrophilic, basic)

F, Y, W (hydrophobic, aromatic)

L, I, V, M (hydrophobic)

C (cross-link forming)

In the most preferred embodiment the present invention relates to the polymorphism or mutation of the MASP-2 gene which causes the substitution of Asp in position 105 to Gly in MASP-2 and/or MAp-19.

Any amino acid substitutions within position 1 to 166 of the MASP-2 or MAp-19 proteins having a similar effect as the Asp105Gly substitution with respect to the MASP-2 activity are covered by this invention.

Preferably the polymorphism of the MASP-2 gene, is the presence of the G allele in position 359 corresponding to the position of the sequence set forth in SEQ ID NO: 3 or 4 or the corresponding allele in the complementary strand or a polymorphism being coupled to said allele.

The effect of a polymorphism may be estimated by quantifying the MASP-2 or MAp-19 amount in a blood sample as described herein.

In one embodiment the polymorphism leads to a reduced MASP-2/MAp-19 concentration in blood, such as to below 75% of normal level, such as to below 50% of normal level, or such as to below 25% of normal level. In the present context the normal level corresponds to the level of MASP-2 or/and MAp-19 in blood samples collected from individuals with normal functional activity of MBL-MASP complexes. It is a preferred embodiment of the invention that the polymorphism leads to a reduced functional activity MASP-2/MAp-19, such as to below 75% of normal level, such as to below 50% of normal level, or such as to below 25% of normal level. In the present context functional activity of MASP-2/MAp-19 is determined as a capability of MASP-2/MAp-19 to bind to MBL or ficolin and thereby to activate the complement C4 in an in vitro test, such as for example a described below.

Methods of Determining Polymorphism

Many methods are known in the prior art for determining the presence of particular nucleotide sequences or for determining particular proteins having particular amino acid sequences. All of these methods may be adapted for determining the polymorphisms according to the present invention.

One common method for detecting single nucleotide polymorphisms (SNP) comprises the use of a probe bound to a detectable label. By carrying out hybridisation under conditions of high stringency it is ensured that the probe only hybridises to a sequence which is 100% complementary to the probe. According to the present invention this method comprises hybridising a probe to a target nucleotide sequence comprising at least position 359 of SEQ ID NO: 3 or the corresponding position of the complementary strand.

In the scope of the present invention the term “hybridisation” signifies hybridisation under conventional hybridising conditions, preferably under stringent conditions, as described for example in Sambrook et al., Molecular Cloning, A Laboratory Manual, 2nd Edition (1989) Cold Spring Harbor Laboratory Press, Cold Spring Harbor, N.Y. The term “stringent” when used in conjunction with hybridisation conditions is as defined in the art, i.e. 15-20° C. under the melting point Tₘ, cf. Sambrook et al, 1989, pages 11.45-11.49. Preferably, the conditions are “highly stringent”, i.e. 5-10° C. under the melting point Tₘ. Under highly stringent conditions hybridisation only occurs if the identity between the oligonucleotide sequence and the locus of interest is 100%, while no hybridisation occurs if there is just one mismatch between oligonucleotide and DNA locus. Such optimised hybridisation results are reached by adjusting the temperature and/or the ionic strength of the hybridisation buffer as described in the art. However equally high specificity may be obtained using high-affinity DNA analogues. One such high-affinity DNA analogues has been termed “locked oligonucleotide” (LNA). LNA is a novel class of bicyclic oligonucleotide analogues in which the furanose ring conformation is restricted in by a methylene linker that connects the 2′-O position to the 4′-C position. Common to all of these LNA variants is an affinity toward complementary oligonucleotides, which is by far the highest reported for a DNA analogue (Ørum et al. (1999) Clinical Chemistry 45, 1898-1905; WO 99/14226 EXIQON). LNA probes are commercially available from Proligo LLC, Boulder, Colo., USA. Another high-affinity DNA analogue is the so-called protein oligonucleotide (PNA). In PNA compounds, the sugar backbone of an oligonucleotide is replaced with an amide containing backbone, in particular an aminoethylglycine backbone. The nucleobases are retained and are bound directly or indirectly to aza nitrogen atoms of the amide portion of the backbone (Science (1991) 254: 1497-1500).

Various different labels can be coupled to the probe. Among these fluorescent reporter groups are preferred because they result in a high signal/noise ratio.

Suitable examples of the fluorescent group include fluorescein, Cy2, Cy3, Cy3.5, Cy5; Cy5.5, Cy7, acridin, Hoechst 33258, Rhodamine, Rhodamine Green, Tetramethylrhodamine, Texas Red, Cascade Blue, Oregon Green, Alexa Fluor, europium and samarium.

Another type of labels are enzyme tags. After hybridisation to the target oligonucleotide sequence a substrate for the enzyme is added and the formation of a coloured product is measured. Examples of enzyme tags include a beta-Galactosidase, a peroxidase, horseradish peroxidase, a urease, a glycosidase, alkaline phosphatase, chloramphenicol acetyltransferase and a luciferase.

A further group of labels include chemiluminescent group, such as hydrazides such as luminol and oxalate esters.

A still further possibility is to use a radioisotope and detect the hybrid using scintillation counting. The radioisotope may be selected from the group consisting of ³²P, ³³P, ³⁵S, ¹²⁵I, ⁴⁵Ca, ¹⁴C and ³H.

One particularly preferred embodiment of the probe based detection comprises the use of a capture probe for capturing a target oligonucleotide sequence. The capture probe is bound to a solid surface such as a bead, a well or a stick. The captured target oligonucleotide sequence can then be contacted with the detection probe under conditions of high stringency and the allele to be detected.

Other suitable methods include using mass spectrometry, single base extension, determining the Tm profile of a hybrid between a probe and a target oligonucleotide sequence, using single strand conformation polymorphism, using single strand conformation polymorphism heteroduplex, using RFLP or RAPD, using sequencing of a target oligonucleotide sequence from said biological sample.

In connection with several of these methods there is a need for amplifying the amount of a target nucleotide sequence in the biological sample isolated from the subject. Amplification may be performed by any known method including methods selected from the group consisting of polymerase chain reaction (PCR), Ligase Chain Reaction (LCR), Oligonucleotide Sequence-Based Amplification (NASBA), strand displacement amplification, rolling circle amplification, and T7-polymerase amplification.

More particularly PCR based amplification of the DNA sequence encoding the MASP-2/Map-19 polypeptide can be carried out using a primer pair comprising SEQ ID NO: 5 (5′-GCGAGTACGACTTCGTCAAGG-3′) and SEQ ID NO: 6 (5′-CTCGGCTGCATAGAAGGCCTC-3′) to amplify parts of the region of the MASP-2 gene encoding the CUB-1 domain, and/or a primer pair comprising SEQ ID NO: 7 (5′-CCAGACCTGGAAAGTTAGC-3′) and SEQ ID NO: 8 (5′-GGCTCAAGTTCCAAGTATTGC-3′) to amplify part of the region of the MASP-2 gene encoding the CCP2 domain of MASP-2. One of the primers may comprise a moiety for subsequent immobilisation of the amplified fragments.

The polymorphism located in the exon(s) of the MASP-2 gene encoding the CUB1 domain may also be detected by isolating MASP-2 or MAp-19 from a biological sample and determining in the isolated proteins the presence or absence of the Asp in position 105 by sequencing said protein. The mutation may be detected by any conventional methods known in the art, for example by a method selected from the group comprising mass-spectroscopy, gel electrophoresis, MALDI-TOF mass spectroscopy, ELISA, protein arrays.

As the substitution from Asp to Gly causes a (minor) change in molecular weight of the MASP-2 or MAp-19 protein it may be possible to determine the presence of either of these residues by determining the molecular weight of said proteins or peptide fragments derived from said proteins.

A mutation of positively charged Asp to uncharged Gly may cause a change in the overall charge of a protein. Therefore, the mutation of the invention may be detected by conventional methods for estimation of the charge of proteins or peptides, for example by isoelectrofocusing.

A substitution of a single amino acid may in some cases also cause a conformational change in the 3D structure of a protein. The change may take place locally, in a restricted area of a protein, e.g. in the CUB-1 domain of MASP-2 or MAp-19, but it may also influence the overall 3D structure of a protein. Any change in the structure may be detected by different physical methods developed for evaluation of conformational changes of biological molecules, such as for example circular dichroism (CD). The substitution may also be detected by methods developed in the art for analysis of protein function, as a change in the charge or the structural perturbations may lead to a major influence on the protein function. It may also be determined by antibodies raised against conformational or sequence determinates.

Function of the Mutated Proteins

Accordingly, the method for determining a predisposition for a manifestation of immune system related diseases in an individual having a mutation in the MASP-2 gene featured by the present invention comprises in another embodiment analysis of functional capabilities of the mutated proteins.

The MASP-2 and MAp-19 proteins both are essential components of lectin complexes of blood, such as the MBL-complex and the ficolin complex, which in turn are major components of the innate immune defence system. The lectin-MASP complexes comprising MASP-1, MASP-2, MASP-3 and MAp-19, activate the complement system of blood. This activation is partially mediated by the lectin-associated MASP-2, which cleaves the complement factors C4 and C2 thus generating the C3 convertase. Therefore, the method according to the present invention further comprises examining the activity of the MBL-MASP or ficolin-MASP complexes in a biological sample collected from an individual, said activity being determined as an ability of said complexes to activate the C4 complement.

According to the present invention an MBL-MASP or ficolin-MASP complex present in a biological sample collected from an individual having the described above mutation in the MASP-2, said mutation being correlated with an immune system related disease, has impaired capability to activate the C4 complement.

An impaired activity of the MBL-MASP or ficolin-MASP complexes leading to an increased susceptibility to frequent infections may in some cases be dependent on an insufficient enzymatic activity of the individual proteins forming the complexes. Another reason for an impaired activity of the MBL-MASP or ficolin-MASP complexes may be the absence or an insufficient amount of an individual protein of the complex. Thus, the method for determining a predisposition for a manifestation of immune system related diseases in an individual presented by the invention comprises the further examining of the protein composition of the MBL-complexes and the ficolin-complexes in a biological sample collected from said individual.

Analysis of protein composition of a multiprotein complex may be done by using a combination of different conventional methods well known in the art. For example, an MBL complex or a ficolin-complex may be separated into different components by PAGE with further analysis of the individual components by immunoblotting. Otherwise, it may be done by a quantitative time resolved immunoflourimetric assay (TRIFMA) comprising the steps of 1) coating microtitre wells with 1 μg anti-C′MASP-2 antibody; 2) blocking with Tween-20; 3) applying test samples, e.g. diluted plasma or serum samples: 4) applying Eu-labelled anti-N′MASP-2 antibody; 5) applying enhancement solution (Wallac Ltd): 6) reading the Eu on a time resolved fluorometer. Similarly, the estimation may also be carried out by using ELISA implying the following modifications of the above assay: biotin-labelled anti-N′MASP-2 on step 4, alkaline phosphatase-labelled avidin on step 5, a standard alkaline phosphatase substrate on step 5 and reading the intensity of the colour on step 6. Between each step, the plate is to be incubated at room temperature and washed, except between step 6 and 7. A calibration curve may be constructed using dilutions of pooled normal plasma, arbitrarily set to contain 1 unit of MASP-2 per ml. The microtitre wells may alternatively be coated with antibodies against MBL or ficolin or with a ligand for MBL or ficolin, e.g. polysaccharide.

Examination of capability of the MBL-MASP/ficolin-MASP complexes to activate the complement system may be estimated by its capacity to activate the C4 complement. When C4 is cleaved by MASP-2 an active thiol ester is exposed and C4 becomes covalently attached to nearby nucleophilic groups. A substantial part of the C4b will thus become attached to the coated plastic well and may be detected by an anti-C4 antibody. Thus, assays of the functional activity of MASP-2 either alone or as part of a lectin complex may be performed by several methods. The activity of MBL/ficolin-MASP-2 to cleave C4 may be assayed by the following method for detecting MASP-2, said method comprising an assay for MASP-2 activity, comprising the steps of 1) applying a sample comprising MBL (a predetermined amount) or ficolin to a solid phase obtaining the bound MBL, 2) applying MASP-2 (a predetermined amount) to the bound lectin, 3) applying at least one complement factor to the complexes, 3) detecting the amount of cleaved complement factors, 4) correlating the amount of cleaved complement factors to the amount of MASP-2, and 5) determining the activity of MASP-2. Otherwise it may be done by 1) applying a sample comprising of MBL/ficolin-MASP-2 complexes (a predetermined amount) to a solid phase obtaining the bound complexes, 2) applying at least one complement factor to the complexes, 3) detecting the amount of cleaved complement factors, 4) correlating the amount of cleaved complement factors to the amount of complexes, and 5) determining the activity of MASP-2.

A quantitative TRIFMA for MASP-2 activity is constructed by 1) coating microtitre wells with 1 μg mannan in 100 μl buffer; 2) blocking with Tween-20; 3) applying test samples, e.g. diluted plasma or serum samples: 4) applying purified complement factor C4 at 5 μg/ml; 5) incubating for one hour at 37° C.; 6) applying Eu-labelled anti-C4 antibody; 7) applying enhancement solution; and 8) reading the Eu by time resolved fluorometry. (Estimation by ELISA may be carried out similarly, e.g. by applying biotin-labelled anti-C4 in step 6; 7) applying alkaline phosphatase-labelled avidin; 8) applying substrate; and 9) reading the colour intensity). Between each step the plate is to be incubated at room temperature and washed, except between step 7 and optionally step 8 and 9. A calibration curve can be constructed using dilutions of one selected normal plasma, arbitrarily said to contain 1 unit of MBL/MASP-2 activity per ml. The assay may further be carried out at conditions, which preclude activation of C4 by the classical or alternative complement activation pathways. Inhibition of the classical complement pathway may, when desired, be done by carrying out the assay at high ionic strength, such as wherein the salt concentration is above 0.2 M, such as above 2.5 M, such as in the range of from 0.3 M to 10 M, such as from 0.5 M to 5 M, such as from 0.7 M to 2 M, such as from 0.9 M to 2 M, such as about 1.0 M. The salts used may be any one or more salts suitable for the assay, such as salts selected from NaCl, KCl, MgCl₂, CaCl₂, NaI, KCl, MgI₂, CaI₂, from NaBr, KBr, MgBr₂, CaBr₂, Na₂S₂O₃, (NH₄)₂SO₄, and NH₄HCO₃. The inhibition of the classical pathway does generally not interfere with the MBL/MASP complex but destroys the C1qrs complex. Inhibition of the classical pathway may also be carried out by adding antibodies against the C1 complex or the other inhibitors of the classical pathway.

The inhibition of the alternative pathway may be carried out by diluting the sample at least 5 times, such as at least 10 times, such as at least 20 times or more, before conducting the assay. It may also be carry out by adding inhibitors selective for the alternative pathway.

The estimation of free MASP-2/MAp-19 in samples from individuals is carried out by first removing MBL/MASP-1 and MBL/MASP-2 complexes by incubating with Sepharose-coupled mannan (300 μl of 10 fold diluted plasma or serum is incubated with 10 μl beads), and then analyzing the supernatant. The assay may be carried out in the TRIFMA format and proceeds as follows: 1) coating microtitre wells with 1 μg mannan in 100 μl buffer; 2) blocking with Tween-20; 3) incubating a sample at a 1000 fold dilution in buffer with 100 ng of MASP-free MBL/ml, and applying 100 μl of the mixture per well; 4) incubating over night at 4° C.; 4) washing and applying purified complement factor C4 at 5 μg/ml; 5) incubating for one hour at 37° C.; 6) applying Eu-labelled anti-C4 antibody; 7) applying enhancement solution; and 8) reading the Eu by time resolved fluorometry. (Estimation by ELISA may be carried out similarly implying the above described modifications). A calibration curve may be constructed using dilutions of an MBL-deficient plasma sample, arbitrarily said to contain 1 unit of MASP-2 activity per ml. The assay is carried out at conditions, which preclude activation of C4 by the classical or alternative complement activation pathways (see above).

The concentration of MASP-2 in blood my be determined by a sandwich technique using antibodies directed to MASP-2.

According to the present invention the predisposition to manifestation of immune system related diseases is determined by the relative absence of the MASP-2 (SEQ ID NO: 1) and/or MAp19 (SEQ ID NO: 2) proteins in the MBL-complexes separated from a biological sample collected from an individual.

Biological Sample

The biological sample used in the present invention may be any suitable biological sample comprising genetic material and/or proteins forming an MBL complex. In a preferred embodiment the sample is a blood sample, a plasma sample, as serum sample, a tissue sample, a secretion sample, semen, ovum, hairs, nails, tears, and urine. The most convenient sample type is a blood sample, a plasma sample or a serum sample.

Oligo- and Polynucleotides

In one aspect the invention relates to an isolated oligonucleotide comprising at least 10 contiguous nucleotides being 100% homologous to a subsequence of the MASP-2 gene comprising or adjacent a polymorphism or mutation being correlated to an immune-related disease. Such probes may be used for detecting the presence of a polymorphism of interest and they may form part of a gene therapy vector used for treating the immune system related diseases.

Preferably the isolated oligonucleotide comprises at least 10 contiguous bases of the sequence comprising bases 375 to 385 of SEQ ID NO: 3 or the corresponding complementary strand or from a strand sharing at least 90% sequence identity more preferably at least 95% sequence identity with SEQ ID NO: 3 or its complementary strand. These particular probes may be used for assessing the polymorphism in the MASP-2 gene, which is strongly correlated with immune-related diseases.

The length of the isolated oligonucleotide depends on the purpose. The length may be at least 15 contiguous nucleotides, such as at least 20 nucleotides. An upper limit preferably determines the maximum length of the isolated oligonucleotide. Accordingly, the isolated oligonucleotide may be less than 250 nucleotides, more preferably less than 150 nucleotides, more preferably less than 100 nucleotides, such as less than 75 nucleotides, for example less than 50 nucleotides, such as less than 40 nucleotides, for example less than 30 nucleotides, such as less than 20 nucleotides.

The isolated oligonucleotide may comprise from 10 to 50 nucleotides, such as from 10 to 15, from 15 to 20, from 20 to 25, or comprising from 20 to 30 nucleotides, or from 15 to 25 nucleotides.

Depending on the use a polymorphism may be located in the centre of the oligonucleotide sequence, in the 5′ end part of the oligonucleotide sequence, or in the 3′ end part of the oligonucleotide sequence.

For detection based on single base extension the sequence of the oligonucleotide is adjacent the mutation/polymorphism, either in the 3′ or 5′ direction.

The sequence of an oligonucleotide may be complementary to a sub-sequence of the coding strand of a target nucleotide sequence or to a sub-sequence to the noncoding strand of a target nucleotide sequence as the polymorphism may be assessed with similar efficiency in the coding and the non-coding strand.

The isolated oligonucleotide may be made from RNA, DNA, LNA, PNA monomers or from chemically modified nucleotides capable of hybridising to a target oligonucleotide sequence. The oligonucleotides may also be made from mixtures of said monomers.

In another aspect the invention concerns an oligonucleotide molecule comprising more then 250 contiguous nucleotides. Such an oligonucleotide molecule is referred herein as a polynucleotide.

In one embodiment, the invention relates to a polynucleotide encoding the MASP-2 polypeptide having the sequence identified as SEQ ID NO: 1. In another embodiment the invention concerns a polynucleotide the MASP-2 polypeptide wherein Gly at a position corresponding to position 105 of the amino acid sequence set forth in SEQ ID NO: 1 is substituted for Asp. In still another embodiment the invention relates to a polynucleotide encoding the MAp-19 polypeptide having the sequence identified as SEQ ID NO: 2. In yet another embodiment the invention relates to a polynucleotide encoding the MAp-19 polypeptide wherein Gly in position corresponding to position 105 of the amino acid sequence set forth in SEQ ID NO: 2 is substituted for Asp.

The polynucleotides encoding MASP-2 and MAp-19 may be cDNA, genomic DNA, synthetic DNA, or RNA, and may be double-stranded or single-stranded (i.e., either a sense or an antisense strand). Fragments of these polynucleotides are also considered within the scope of the invention, and can be produced, for example, by the polymerase chain reaction (PCR) or generated by treatment with one or more restriction endonucleases. A ribonucleic acid (RNA) molecule can be produced by in vitro transcription.

The polynucleotide molecules of the invention may contain naturally occurring sequences, or sequences that differ from those that occur naturally, but, due to the degeneracy of the genetic code, encode the same polypeptide. In addition, these polynucleotide molecules are not limited to sequences that only encode polypeptides, and thus, can include some or all of the non-coding sequences that lie upstream or downstream from a coding sequence.

In a preferred embodiment the invention relates to an isolated polynucleotide molecule encoding the polypeptide defined herein, the molecule comprising a nucleotide sequence encoding a polypeptide having sequence that is at least 60% identical to the sequence of SEQ ID NO: 1 or 2, said sequences having a substitution of Asp in position 105 for another amino acid. The polypeptide is preferably the MASP-2 protein having a sequence at least 60% identical to SEQ ID NO: 1 or the MAp-19 protein having a sequence at least 65% identical to SEQ ID NO: 2, wherein said proteins are carrying the mutation Asp Gly in position 105. It is more preferable if the above identity of the sequences is at least 65%, more preferably is least 70%, even more preferably is at least 75%, even more preferably at least 80%, even more preferably at least 85%, even more preferably at least 90%, and the most preferably at least 95%.

In another preferred embodiment the invention relates to an isolated oligo- or polynucleotide sequence encoding a polypeptide comprising an epitope, which is recognised by an antibody of the invention described below. Such an oligo- or polynucleotide preferably encodes a polypeptide comprising a fragment of any of the amino acid sequences set forth in SEQ ID NOS: 1 or 2 from amino acid 1 to 160, said fragment comprising a mutation which is essential for recognition said polypeptides by the antibodies. By the term “essential” in the present context is meant that the mutation is either comprised by an amino acid sequence creating a new unique epitope on the mutated polypeptide absent on the corresponding non-mutated polypeptide, or due to major structural perturbations of the 3D structure of the mutated polypeptide lead to creation of a new unique epitope located distantly from the mutated position. According to the invention the preferred oligo- or polynucleotides are those that encode fragments of MASP-2 and MAp-19 polypeptides having Gly in position 105.

Preferred oligonucleotides of the invention are also those that encode a region of non-mutated MASP-21MAp-19 that comprise Asp in position 105. Polypeptides and peptide fragments encoded by such nucleotide sequences are in the scope of preferences of the invention as they may be used for the production of antibodies, which are discussed further in the text, or the preparation of a medicament, which is discussed further in the text.

The oligonucleotides and polynucleotide molecules of the invention can be synthesized (for example, by phosphoramidite-based synthesis) or obtained from a biological cell, such as the cell of a mammal. Thus, the oligonucleotides and/or polynucleotides can be those of a human, mouse, rat, guinea pig, cow, sheep, horse, pig, rabbit, monkey, dog, or cat. Combinations or modifications of the nucleotides within these types of oligonucleotides and polynucleotides are also encompassed.

In addition, the isolated oligonucleotide and polynucleotide molecules of the invention encompass fragments that are not found as such in the natural state. Thus, the invention encompasses recombinant molecules, such as those in which a oligonucleotide or polynucleotide molecule (for example, an isolated polynucleotide molecule encoding the mutated MASP-2 or mutated MAp-19) is incorporated into a vector (for example, a plasmid or viral vector) or into the genome of a heterologous cell (or the genome of a homologous cell, at a position other than the natural chromosomal location). Recombinant oligonucleotide and polynucleotide molecules and uses thereof are discussed further below.

In the event the oligonucleotide or polynucleotide molecules of the invention encode or act as antisense molecules, they can be used for example, to regulate translation of the mutated MASP-2 or MAp-19. Techniques associated with detection or regulation of oligonucleotide or polynucleotide expression are well known to skilled artisans and can be used to diagnose and/or treat disorders associated with occurrence of the described mutation of the MASP-2 gene. These oligonucleotide and polynucleotide molecules are discussed further below in the context of their clinical utility.

The invention also encompasses oligonucleotide and polynucleotide molecules that hybridize under stringent conditions to a polynucleotide molecule encoding the MASP-2 or MAp-19 polypeptide. The cDNA sequence described herein (SEQ ID NO: 3) can be used to identify these oligonucleotides or polynucleotides, which include, for example, sequences that encode homologous polypeptides in other species, and splice variants of the MASP-2 gene in humans or other mammals. Accordingly, the invention features methods of detecting and isolating these oligonucleotide and polynucleotide molecules.

Using these methods, a sample (for example, a polynucleotide library, such as a cDNA or genomic library) is contacted (or “screened”) with a MASP-2-specific probe, for example, a fragment of SEQ ID NO: 3 that is at least 12 nucleotides long. The probe will selectively hybridize to oligonucleotides or polynucleotide encoding related polypeptides (or to complementary sequences thereof). Since the polypeptide encoded by a MASP-2 polynucleotide is related to other serine proteases, the term “selectively hybridize” is used to refer to an event in which a probe binds to oligonucleotides encoding the MASP-2 sequence or fragments thereof (or to complementary sequences thereof) to a detectably greater extent than to oligo- or polynucleotides encoding other serine proteases (or to complementary sequences thereof). The probe, which may contain at least 12 (for example, 15, 25, 50, 100, or 200 nucleotides) can be produced using any of several standard methods (see, for example, Ausubel et al., “Current Protocols in Molecular Biology, Vol. I,” Green Publishing Associates, Inc., and John Wiley & Sons, Inc., NY, 1989). For example, the probe can be generated using PCR amplification methods in which oligonucleotide primers are used to amplify a MASP-2-specific oligonucleotide sequence (for example, a oligonucleotide encoding the N-terminus of mature MASP-2) that can be used as a probe to screen the library and detect oligonucleotide sequences (within the library) that hybridize to the probe.

One single-stranded oligonucleotide or polynucleotide is said to hybridize to another if a duplex forms between them. This occurs when one oligo- or polynucleotide contains a sequence that is the reverse and complement of the other (this same arrangement gives rise to the natural interaction between the sense and antisense strands of DNA in the genome and underlies the configuration of the “double helix”). Complete complementarity between the hybridizing regions is not required in order for a duplex to form; it is only necessary that the number of paired bases is sufficient to maintain the duplex under the hybridization conditions used.

In one aspect, the invention relates to a oligonucleotide probe capable of forming a complex with the MASP-2-encoding nucleotide sequence under stringent conditions, such as a sequence capable of hybridizing to a nucleotide sequence which is at least 80% identical to SEQ ID NO: 3.

A probe for hybridization may be an anti-sense oligonucleotide with respect to a nucleotide sequence encoding MASP-2.

Typically, hybridization conditions are of low to moderate stringency. These conditions favour specific interactions between completely complementary sequences, but allow some non-specific interaction between less than perfectly matched sequences to occur as well. After hybridization, the oligonucleotides can be “washed” under moderate or high conditions of stringency to dissociate duplexes that are bound together by some non-specific interaction (the oligonucleotides that form these duplexes are thus not completely complementary).

As is known in the art, the optimal conditions for washing are determined empirically, often by gradually increasing the stringency. The parameters that can be changed to affect stringency include primarily temperature and salt concentration. In general, lower the salt concentration and higher the temperature, the higher the stringency. Washing can be initiated at a low temperature (for example, room temperature) using a solution containing a salt concentration that is equivalent to or lower than that of the hybridization solution. Subsequent washing can be carried out using progressively warmer solutions having the same salt concentration. As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where oligonucleotides or polynucleotides are hybridised, the conditions used to achieve a given level of stringency will vary. There is not one set of conditions, for example, that will allow duplexes to form between all oligonucleotides or polynucleotide that are 85% identical to one another; hybridisation also depends on unique features of each oligo- or polynucleotide. The length of the sequence, the composition of the sequence (for example, the content of purine-like nucleotides versus the content of pyrimidine-like nucleotides) and the type of the sequence (for example, DNA or RNA) affect hybridization. An additional consideration is whether one of the oligo- or polynucleotides is immobilized (for example, on a filter).

An example of a progression from lower to higher stringency conditions is the following, where the salt content is given as the relative abundance of SSC (a salt solution containing sodium chloride and sodium citrate; 2×SSC is 10-fold more concentrated than 0.2×SSC). Oligonucleotides or polynucleotides are hybridized at 42° C. in 2×SSC/0.1% SDS (sodium dodecylsulfate; a detergent) and then washed in 0.2×SSC/0.1% SDS at room temperature (for conditions of low stringency); 0.2×SSC/0.1% SDS at 42° C. (for conditions of moderate stringency); and 0.1×SSC at 68° C. (for conditions of high stringency). Washing can be carried out using only one of the conditions given, or each of the conditions can be used (for example, washing for 10-15 minutes each in the order listed above). Any or all of the washes can be repeated. As mentioned above, optimal conditions will vary and can be determined empirically.

A second set of conditions that are considered “stringent conditions” are those in which hybridization is carried out at 50° C. in Church buffer (7% SDS, 0.5% NaHPO₄, 1 M EDTA, 1% bovine serum albumin) and washing is carried out at 50° C. in 2×SSC.

Once detected, the oligonucleotide or polynucleotide molecules can be isolated by any of a number of standard techniques (see, for example, Sambrook et al., “Molecular Cloning, A Laboratory Manual,” 2nd Ed. Cold Spring Harbor Laboratory Press, Cold Spring Harbor, N.Y., 1989).

Recombinant Production

The invention also encompasses: (a) expression vectors that contain any of the foregoing MASP-2 or MAp-19-related coding sequences comprising a mutation which has been correlated with an immune system disease and/or their complements (that is, “antisense” sequence); (b) expression vectors that contain any of the foregoing MASP-2 or MAp-19-related coding sequences comprising the above mutation operatively associated with a regulatory element (examples of which are given below) that directs the expression of the coding sequences; (c) expression vectors containing, in addition to sequences encoding a mutated MASP-2 or MAp-19 polypeptide, polynucleotide sequences that are unrelated to polynucleotide sequences encoding MASP-2 or MAp-19, such as molecules encoding a reporter or marker; and (d) genetically engineered host cells that contain any of the foregoing expression vectors and thereby express the polynucleotide molecules of the invention in the host cell. Preferably, the polynucleotides encode polypeptides that, regardless of length, are soluble under normal physiological conditions.

Recombinant polynucleotide molecule may contain a sequence encoding a mutated soluble MASP-2, mature MASP-2, MASP-2 having a signal sequence or functional domains of MASP-2 such as for example a MBL-binding domain. The full-length mutated MASP-2 polypeptide, a domain of MASP-2, or a fragment thereof may be fused to additional polypeptides, as described below. Similarly, the polynucleotide molecules of the invention can encode the mature form of mutated MASP-2 or a form that encodes a polypeptide, which facilitates secretion. In the latter instance, the polypeptide is typically referred to as a proprotein, which can be converted into an active form by removal of the signal sequence, for example, within the host cell. Proproteins can be converted into the active form of the protein by removal of the inactivating sequence.

The regulatory elements referred to above include, but are not limited to, inducible and non-inducible promoters, enhancers, operators and other elements, which are known to those skilled in the art, and which drive or otherwise regulate gene expression. Such regulatory elements include, but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast—mating factors.

Similarly, the polynucleotide can form part of a hybrid gene encoding additional polypeptide sequences, for example, sequences that function as a marker or reporter. Examples of marker or reporter genes include lactamase, chloramphenicol acetyltransferase (CAT), adenosine deaminase (ADA), aminoglycoside phosphotransferase (neo^{r}, G418^{r}), dihydrofolate reductase (DHFR), hygromycin-Bphosphotrans-ferase (HPH), thymidine kinase (TK), lacZ (encoding-galactosidase), green fluorescent protein (GFP), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter. Generally, the hybrid polypeptide will include a first portion and a second portion; the first portion being a MASP-2 polypeptide and the second portion being, for example, the reporter described above or an immunoglobulin constant region, or the flow of portions of the first polypeptide and the second polypeptide in a hybrid polypeptide sequence may be reverted to the above.

The expression systems that may be used for purposes of the invention include, but are not limited to, microorganisms such as bacteria (for example, E. coli and B. subtilis) transformed with recombinant bacteriophage DNA, plasmid DNA, or cosmid DNA expression vectors containing the polynucleotide molecules of the invention; yeast (for example, Saccharomyces and Pichia) transformed with recombinant yeast expression vectors containing the polynucleotide molecules of the invention (preferably containing the polynucleotide sequence of MASP-2 (SEQ ID NO:3) or the polynucleotide sequence of MAp-19 (SEQ ID NO: 4); insect cell systems infected with recombinant virus expression vectors (for example, baculovirus) containing the polynucleotide molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing MASP-2 nucleotide sequences; or mammalian cell systems (for example, COS, CHO, BHK, 293, VERO, HeLa, MDCK, W138, and NIH 3T3 cells) harbouring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors may be advantageously selected depending upon the use intended for the gene product being expressed. For example, when a large quantity of such a protein is to be produced, for the generation of pharmaceutical compositions containing MASP-2 and/or MAp-19 polypeptides or for raising antibodies to those polypeptides, vectors that are capable of directing the expression of high levels of fusion protein products including peptides that are readily purified may be desirable. Such vectors include, but are not limited to, the E. coli expression vector pUR278 (Ruther et al., EMBO J. 2:1791, 1983), in which the coding sequence of the insert may be ligated individually into the vector in frame with the lacZ coding region so that a fusion protein is produced; pIN vectors (Inouye and Inouye, Oligonucleotides Res. 13:3101-3109, 1985; Van Heeke and Schuster, J. Biol. Chem. 264:5503-5509, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) can be used as a vector to express foreign genes. The virus grows in Spodoptera frugiperda cells. The coding sequence of the insert may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (for example, see Smith et al., J. Virol. 46:584, 1983; Smith, U.S. Pat. No. 4,215,051).

In mammalian host cells, a number of viral-based expression systems may be utilized. In cases where an adenovirus is used as an expression vector, the polynucleotide molecule of the invention may be ligated to an adenovirus transcription/translation control complex, for example, the late promoter and tripartite leader sequence forming a chimeric gene. This chimeric gene may then be inserted in the adenovirus genome by in vitro or in vivo recombination. Insertion in a non-essential region of the viral genome (for example, region E1 or E3) will result in a recombinant virus that is viable and capable of expressing a MASP-2 gene product in infected hosts (for example, see Logan and Shenk, Proc. Natl. Acad. Sci. USA 81:3655-3659, 1984). Specific initiation signals may also be required for efficient translation of inserted polynucleotide molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544, 1987).

In addition, a host cell strain may be chosen, which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. The mammalian cell types listed above are among those that could serve as suitable host cells.

For long-term, high-yield production of recombinant proteins, stable expression is preferred. For example, cell lines which stably express the MASP-2 sequences described above may be engineered. Rather than using expression vectors which contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express MASP-2. Such engineered cell lines may be particularly useful in screening and evaluation of compounds that affect the endogenous activity of the gene product and for production of MASP-2 for theraputic uses. These methods may also be used to modify cells that are introduced into a host organism either for experimental or theraputic purposes. The introduced cells may be transient or permanent within the host organism.

A number of selection systems can be used. For example, the herpes simplex virus thymidine kinase (Wigler, et al., Cell 11:223, 1977), hypoxanthine-guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., Proc. Natl. Acad. Sci. USA 77:3567, 1980; O'Hare et al., Proc. Natl. Acad. Sci. USA 78:1527, 1981); gpt, which confers resistance to mycophenolic acid (Mulligan and Berg, Proc. Natl. Acad. Sci. USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984).

Alternatively, any fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

Isolated Polypeptides and Peptide Fragments

Isolated Polypeptides

The invention further concerns isolated polypeptides and peptide fragments related to immune system diseases correlated with a mutation of the MASP-2 gene.

Isolated polypeptides of the invention described herein are those encoded by any of the oligo- or polynucleotide molecules described above and include MASP-2 and/or MAp-19 fragments, mutants, truncated forms, and fusion proteins. These polypeptides can be prepared for a variety of uses, including but not limited to the generation of antibodies, as reagents in diagnostic assays, for the identification of other cellular gene products or compounds that can modulate the MBL-complement pathway, and as pharmaceutical reagents useful for the treatment of inflammation and certain disorders (described below) that are associated with activity of the lectin pathway. Preferred polypeptides are substantially pure MASP-2 or MAp-19 polypeptides which carry a mutation in any parts of amino acid comprising a MBL-binding region, said mutation capable of inhibiting binding MASP-2 or MAp-19 to MBL, including those polypeptides that correspond to the polypeptide with an intact signal sequence, the mature form of the polypeptide of the human MASP-2 polypeptide as well as polypeptides representing a part of the MASP-2 polypeptide. Especially preferred are polypeptides that are soluble under normal physiological conditions.

By the term “substantially pure” in the present content is meant a polypeptide which is distinct from any naturally occuring composition, and suitable for at least one of the uses proposed herein. While preparations that are only slightly altered with respect to naturally occuring substances may be somewhat useful, more typically, the preparations are at least 10% by weight (dry weight) the compound of interest. Preferably, the preparation is at least 60%, more preferably at least 75%, and most preferably at least 90%, by weight the compound of interest. Purity can be measured by any appropriate standard method, for example, by column chromatography, polyacrylamide gel electrophoresis, or HPLC analysis.

In particular the invention relates to polypeptides comprising an amino acid sequence identified as SEQ ID NO: 1, wherein Gly in position 105 is substituted for Asp, or a functional equivalent thereof, and/or an amino acid sequence identified as SEQ ID NO: 2, wherein Gly in position 105 is substituted for Asp, or a functional equivalent thereof.

By the term “functional equivalent” in the present context is meant a polypeptide
i) comprising an amino acid sequence having at least 60% identity with the sequence set forth in SEQ ID NO: 1 or 2,
ii) capable of at least one of the functional activities of naturally occurring forms of MASP-2 or MAp-19 in a biological system,
iii) having an impaired binding capability to MBL as compared to non-mutated naturally occurring forms of MASP-2 or MAp-19, and
iv) being recognised by an antibody raised against the MASP-2/MAp-19 polypeptide or a fragment thereof, said polypeptide or fragment carrying the mutation Asp105Gly.

The functional equivalent is preferably a mutant of the MASP-2 protein having a sequence at least 60% identical to SEQ ID NO: 1 or the MAp-19 protein having a sequence at least 65% identical to SEQ ID NO: 2, wherein said proteins are carrying the mutation Asp→Gly in position 105. It is more preferable if the above identity of the sequences is at least 65%, more preferably is least 70%, even more preferably is at least 75%, even more preferably at least 80%, even more preferably at least 85%, even more preferably at least 90%, and the most preferably at least 95%.

Comparisons of functional capabilities of a protein and its functional equivalent are generally based on an assay of biological activity in which equal concentrations of the above polypeptides are used and compared. The comparison can also be based on the amount of the polypeptide required to reach 50% of the maximal activity obtainable.

Functionally equivalent proteins can be those, for example, that contain additional or substituted amino acid residues. Substitutions may be made on the basis of similarity in polarity, charge, solubility, hydrophobicity, hydrophilicity, and/or the amphipathic nature of the residues involved. Amino acids that are typically considered to provide a conservative substitution for one another are specified in the summary of the invention. D-amino acids may be introduced in order to modify the half-life of the polypeptide.

Polypeptides that are functionally equivalent to MASP-2 having the described above mutation can be made using random mutagenesis techniques well known to those skilled in the art (and the resulting MASP-2 proteins carrying additional mutations can be tested for activity). It is more likely, however, that such polypeptides will be generated by site-directed mutagenesis (again using techniques well known to those skilled in the art). These polypeptides may have an enhanced function, i.e., an ability to activate the C4 complement independently of their capability of binding to MBL. Such polypeptides can be used to enhance a potency of the innate system by supplementing the activity of the lectin-complement pathway.

To design functionally equivalent polypeptides, it is useful to distinguish between conserved positions and variable positions. This can be done by aligning the sequence of MASP-2 cDNAs that were obtained from various organisms. Skilled artisans will recognize that conserved amino acid residues are more likely to be necessary for preservation of function. Thus, it is preferable that conserved residues are not altered. Such a conserved residue of the invention is Gly in position 105 of the mutant MASP-2 or MAP-19.

Additional to the above described mutations within the MASP-2 coding sequence new mutations can be made to generate MASP-2 derived polypeptides that are better suited for expression in a selected host cell. Introduction of a glycosylation sequence can also be used to generate a MASP-2 polypeptide with altered biological characteristics.

The polypeptides of the invention can be expressed fused to another polypeptide, for example, a marker polypeptide or fusion partner. For example, the polypeptide can be fused to a hexa-histidine tag to facilitate purification of bacterially expressed protein or a hemagglutinin tag to facilitate purification of protein expressed in eukaryotic cells. The MASP-2 or MAp-19 polypeptide of the invention, or a portion thereof, can also be altered so that it has a longer circulating half-life by fusion to an immunoglobulin Fc domain (Capon et al., Nature 337:525-531, 1989). Similarly, a dimeric form of the MASP-2 polypeptide can be produced, which has increased stability in vivo.

The polypeptides of the invention can be chemically synthesized (for example, see Creighton, “Proteins: Structures and Molecular Principles,” W.H. Freeman & Co., NY, 1983), or, perhaps more advantageously, produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984).

Isolated Peptide Fragments

An important objection of the invention is to provide peptide fragments, which are of relevance to an immune system disease diagnosed in relation to a mutation in the MASP-2 gene.

Isolated peptide fragments of the invention described herein are those that are derived from the sequence set forth in SEQ ID NO: 1. It is a preferred embodiment that said fragments are derived from a part of the SEQ ID NO: 1, which covers the region from 1 to 160 amino acid residue.

Peptide fragments of the invention may be of various size contiguous amino acid sequences. Preferred size of a peptide fragment of the invention lies in a range from 5 to 160 amino acids.

The invention specifically features peptide fragments that
i) are derived from the sequence set forth in SEQ ID NO: 1,
ii) have a size of 5-160 amino acids and
iii) comprise a sequence of at least 5 contiguous amino acids, said sequence corresponding to amino acid residues no. 100-105, 101-106, 102-107, 103-108, 104-109 and/or 105-110 of the sequence set forth in SEQ ID NO: 1.

Other peptide fragments of a special attention of the invention are those that
i) are derived from the sequence set forth in SEQ ID NO: 1,
ii) have a size of 5-160 amino acids,
iii) comprise a sequence of at least 5 contiguous amino acids, said sequence corresponding to amino acid residues no. 100-105, 101-106, 102-107, 103-108, 104-109 and/or 105-110 of the sequence set forth in SEQ ID NO: 1 and
iv) have Gly in position 105 corresponding to the SEQ ID NO: 1.

The invention features also uses the above peptide fragments and polypeptides. Thus, in one embodiment they may be used for the inhibiting the lectin-complement pathway. In another embodiment they may be used for the preparation of a medicament for treatment pathologic conditions associated with undesirably high activity of the lectin-complement pathway. In still another embodiment they may be used for preparation of antibodies capable to selectively binding to an epitope within the MASP-2 or MAp-19 polypeptide comprising Gly in position 105 according to SEQ ID NO: 1 or SEQ ID NO: 2.

Antibody

It is an objective of the present invention to provide an antibody capable of selectively binding to a MASP-2 or MAp-19 derived polypeptide carrying the mutation Asp105Gly, or a functional homologue thereof.

In one embodiment the invention relates to an antibody capable of binding to an epitope comprising a fragment of the amino acid sequence set forth SEQ ID NO: 1 or SEQ ID NO: 2, said fragment containing Gly in position 105 according to SEQ ID NO: 1 or SEQ ID NO: 2. Preferably that the epitope is located within the MASP-2 or MAp-19 proteins.

In another embodiment the invention features an antibody, which is capable of binding to a novel epitope created within the MASP-2 or MAp-19 proteins due to a mutation of the DNA encoding said proteins. The invention preferably refers to a mutation of at least one nucleotide within the coding sequence of the human MASP-2 gene. In a more preferred embodiment, the polymorphism concerns a mutation located in the sequence encoding a fragment of MASP-2 consisting of CUB1, EGF, CUB2, CCP1 and CCP2 domains. More preferably the polymorphism is located in the sequence encoding the CUB1, EGF, CUB2 and CCP1 domains of MASP-2. Even more preferably the polymorphism is a mutation in of the coding sequence of the CUP1 and EGF domains of MASP-2 and/or MAp-19. It is further preferably, if the polymorphism is located in the coding sequence of the CUB1 domain of MASP-2 and/or MAp-19. The preferred position of the mutation in the CUB1 encoding sequence is between nucleotides no. 319 and 399 corresponding to the positions of the sequences set for in SEQ ID NO: 3 or 4. In the most preferred embodiment the method relates to a single nucleotide substitution/mutation A→G in position 359.

In particular the invention relates to an antibody directed against a novel epitope created within MASP-2 or MAp-19 due to the mutation Asp105Gly according to the amino acid sequence identified as SEQ ID NO: 1 or 2.

Additionally, invention relates to an antibody capable of recognition of an epitope located within a region of MASP-2 or MAp-19 comprising Gly in position corresponding to position 105 of the amino acid sequence identified in SEQ ID NO: 1 or 2.

MASP-2 derived polypeptides and peptide fragments comprising Asp or Gly in position 105 according to SEQ ID NO: 1 (or immunogenic fragments or analogs) can be used to raise antibodies useful in the invention; such polypeptides can be produced by recombinant techniques as described the above or synthesized (see, for example, “Solid Phase Peptide Synthesis,” supra; Ausubel et al., supra). In general, the peptides can be coupled to a carrier protein, such as KLH, as described in Ausubel et al., supra, mixed with an adjuvant, and injected into a host mammal. Also the carrier could be PPD. Antibodies can be purified by peptide antigen affinity chromatography.

In particular, various host animals can be immunized by injection with the mutated MASP-2 protein of the invention or a fragment thereof comprising Gly in position 105 according to SEQ ID NO: 1. Host animals include rabbits, mice, guinea pigs, rats, and chickens. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Potentially useful human adjuvants include BCG (bacille Calmette-Guerin) and Corynebacterium parvum. Immunizations may also be carried out by the injection of DNA encoding the mutated MASP-2 or fragments thereof corresponding to the mutated area. Polyclonal antibodies are heterogeneous populations of antibody molecules that are contained in the sera of the immunized animals.

The invention preferably relates to an antibody produced by administering the MASP-2 polypeptide carrying the above described mutation, or part of the such MASP-2 polypeptide, or DNA encoding any such polypeptide, to an animal with the aim of producing antibody. It is preferred that said antibody selectively binds to an epitope on MASP-2 molecule comprising Gly in position 105 according to SEQ ID NO: 1. Another preference of the invention is that said antibody distinguishes the unique epitope on the mutated MASP-2 or MAp-19 polypeptide which has been created by the said mutation.

Antibodies within the invention therefore include polyclonal antibodies and, in addition, monoclonal antibodies, humanized or chimeric antibodies, single chain antibodies, Fab′ fragments, F(ab′)₂ fragments, and molecules produced using a Fab expression library, and antibodies or fragments produced by phage display techniques.

Monoclonal antibodies, which are homogeneous populations of antibodies to a particular antigen, can be prepared using the MASP-2 proteins described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, NY, 1981; Ausubel et al., supra).

In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. (In the case of chckens, the immunoglobulin class can also be IgY.) The hybridoma producing the mAb of this invention may be cultivated in vitro or in vivo. The ability to produce high titers of mAbs in vivo makes this the presently preferred method of production, but in some cases, in vitro production will be preferred to avoid introducing cancer cells into live animals, for example, in cases where the presence of normal immunoglobulins coming from the acitis fluids are unwanted, or in cases involving ethical considerations.

Once produced, polyclonal, monoclonal, or phage-derived antibodies are tested for specific recognition of the above described epitope by Western blot or immunoprecipitation in samples containing the mutated MASP-2 or MAp-19 polypeptides or fragments thereof, e.g., as described in Ausubel et al., supra. Antibodies that specifically recognize and bind to the mutated MASP-2 are useful in the invention. For example, such antibodies can be used in an immunoassay to monitor the level of the mutated MASP-2 in an individual.

In some cases it may be desirable to minimize the potential problems of low affinity or specificity of antisera. In such circumstances, two or three fusions can be generated for each protein, and each fusion can be injected into at least two animals. Antisera can be raised by injections in a series, preferably including at least three booster injections. Spleen cells from the immunised animals may be used for generating monoclonal antibodies.

The antibodies can be used, for example, in the detection of the mutated MASP-2 or MAp-19 in a biological sample as part of a diagnostic assay. Antibodies also can be used in a screening assay to measure the effect of a designed compound on expression of the mutated MASP-2. Thus, the antibody may be coupled to a compound comprising a detectable marker for diagnostic purposes. The makers or labels may be selected from the described above.

In addition, techniques developed for the production of “chimeric antibodies” (Morrison et al., Proc. Natl. Acad. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778, 4,946,778, and 4,704,692) can be adapted to produce single chain antibodies against a MASP-2 related protein or polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab′ fragments that can be generated by reducing the disulfide bridges of F(ab′)₂ fragments. Alternatively, Fab′ expression libraries can be constructed (Huse et al., Science, 246:1275, 1989) to allow rapid and easy identification of monoclonal Fab′ fragments with the desired specificity.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

Kit

In one aspect there is provided a kit for predicting an increased risk of a subject for developing immune related diseases comprising at least one probe comprising a polynucleotide sequence as defined in the previous section. The probe is in one embodiment linked to a detectable label.

In another aspect based on single nucleotide extension the kit further comprises at least one nucleotide monomer labelled with a detectable label, a polymerase and suitable buffers and reagents.

The kit preferably also comprises set of primers for amplifying a region of the MASP-2 gene said region comprising position 359 of SEQ ID NO: 3 or the corresponding complementary strand.

In still another aspect the kit of the invention comprises at least one probe comprising the described above antibody.

The methods described above in which anti-mutated MASP-2-antibodies are employed may be performed, for example, by utilizing pre-packaged diagnostic kits comprising at least one antibody reagent described herein, which may be conveniently used, for example, in clinical settings, to diagnose patients exhibiting symptoms of the disorders described below. Antibodies of the kit can conventionally be labelled, for example an antibody may carry such labels as horse radish peroxidase or alkaline phosphatase, or the antibodies may comprise an fluorescent, luminescent or radioisotope label.

Gene Therapy

Having identified polymorphism(s) as the cause of a disease it is also rendered possible with the present invention to provide a genetic therapy for subjects being diagnosed as having a predisposition according to the invention, said therapy comprising administering to said subject a therapeutically effective amount of a gene therapy vector.

There are various different methods of gene therapy for the subjects defined in the present invention.

The first two are based on activation of the repair system of the cells by introducing into those cells a gene therapy vector which causes “correction” of the polymorphism by presenting the repair mechanism with a template for carrying out the correction. One such type includes the RNA/DNA chimeraplast, said chimeraplast being capable of correcting the polymorphism in cells of said subject. Examples of the design of such chimeraplasts can be found in e.g. U.S. Pat. No. 5,760,012; U.S. Pat. No. 5,888,983; U.S. Pat. No. 5,731,181; U.S. Pat. No. 6,010,970; U.S. Pat. No. 6,211,351.

The second method is based on application of single stranded polynucleotides, wherein the terminal nucleotides protected from degradation by using 3′ and 5′ phosphorothioat-linkage of the monomers. This gene therapy vector is also capable of “correcting” the polymorphism by replacing one nucleotide with another.

These first two types of gene therapy vectors comprise a small sequence (less than 50 bases) which overlaps with the polymorphism in question. Suitable sequences for this purpose are genomic sequences located around the polymorphism.

Other types of gene therapy include the use of retrovirus (RNA-virus). Retrovirus can be used to target many cells and integrate stably into the genome. Adenovirus and adeno-associated virus can also be used. A suitable retrovirus or adenovirus for this purpose comprises an expression construct with the wildtype gene under the control of the wildtype promoter in order to increase the copy number of the wildtype gene.

A further group of gene therapy vectors which are particularly useful for treating subjects with a predisposition according to the present invention against immune system diseases includes vectors comprising interfering RNA (RNAi) for catalytic breakdown of mRNA carrying the polymorphism. RNAi can be used for lowering the expression of a given gene for a relatively short period of time.

Interfering RNA (“RNAi”) is double stranded RNA that results in catalytic degradation of specific mRNAs, and can also be used to lower gene expression. The RNAi vectors of the present invention can suitably be administered to an individual by injection, by gradual infusion over time or by any other medically acceptable mode.

Preferably a gene therapy vector selected from the described above carries a coding DNA sequence for wild type mature MASP-2 and/or Map-19.

MASP-2 is required for the powerful antimicrobial activity of the MBL/MASP complex, and deficiency in MASP-2/Map-19, either genetically determined or acquired, will therefore compromise an individual's resistance to infections and ability to combat established infections. Reconstitution with natural or recombinant MASP-2 using the above described gene therapy is a useful treatment modality in such situations. Recombinant MASP-2 delivered by a gene therapy vector may be in the form of the whole molecule, parts of the molecule, or the whole or part thereof attached by any means to another structure in order to modulate the activity. The recombinant products may be identical in structure to the natural molecule or slightly modified to yield enhanced activity or decreased activity when such is desired.

The infection which may be treated with the above gene therapy vector(s) may be infection by any infectious agent. For example the infection may be caused by a microoroganism. The microoroganism may be represented by a fungi, yeast, bacteria or parasite species.

Bacteria according to the present invention may for example be selected from the group consisting of Achromobacter xylosoxidans, Acinetobacter calcoaceticus, preferably A. anitratus, A. haemolyticus, A. alcaligenes, and A. Iwoffii, Actinomyces israelii, Aeromonas hydrophilia, Alcaligenes species, preferably A. faecalis, A. odorans and A. denitrificans, Arizona hinshawii, Bacillus anthracis, Bacillus cereus, Bacteroides fragilis, Bacteroides melaninogenicus, Bordetella pertussis, Borrelia burgdorferi, Borrelia recurrentis, Brucella species, preferably B. abortus, B. suis, B. melitensis and B. canis, Calymmatobacterium granulomatis, Campylobacter fetus ssp. intestinalis, Campylobacter fetus ssp. jejuni, Chlamydia species, preferably C. psittaci and C. trachomatis, Chromobacterium violaceum, Citrobacter species, preferably C. freundii and C. diversus, Clostridium botulinum, Clostridium perfringens, Clostridium difficile, Clostridium tetani, Corynebacterium diphtheriae, Corynebacterium, preferably C. ulcerans, C. haemolyticum and C. pseudotuberculosis, Coxiella burnetii, Edwardsiella tarda, Eikenella corrodens, Enterobacter, preferably E. cloacae, E. aerogenes, E. hafniae (also named Hafnia alvei) and E. agglomerans, Erysipelothrix rhusiopathiae, Escherichia coli, Flavobacterium meningosepticum, Francisella tularensis, Fusobacterium nucleatum, Gardnerella vaginalis, Haemophilus ducreyi, Haemophilus influenzae, Helicobacter species, Klebsiella species, preferably K. pneumoniae, K. ozaenae og K. rhinoscleromatis, Legionella species, Leptospira interrogans, Listeria monocytogenes, Moraxella species, preferably M. lacunata and M. osloensis, Mycobacterioum bovis, Mycobacterium leprae, Mycobacterium tuberculosis, Mycoplasma species, preferably M. pneumoniae, Neisseria gonorrhoeae, Neisseria meningitidis, Nocardia species, preferably N. asteroides and N. brasiliensis, Pasterurella haemolytica, Pasteurella multocida, Peptococcus magnus, Plesiomonas shigelloides, Pneumococci, Proteus species, preferably P. mirabilis, P. vulgaris, P. reftgeri and P. morganii (also named Providencia rettgeri and Morganella morganii respectively), Providencia species, preferably P. alcalifaciens, P. stuartii and P. rettgeri (also named Proteus rettgeri), Pseudomonas aeruginosa, Pseudomonas mallei, Pseudomonas pseudomallei, Rickettsia, Rochalimaia henselae, Salmonella species, preferably S. enteridis, S. typhi and S. derby, and most preferably Salmonella species of the type Salmonella DT104, Serratia species, preferably S. marcescens, Shigella dysenteriae, S. flexneri, S. boydii and S. sonnei, Spirillum minor, Staphylococcus aureus, Staphylococcus epidermidis, Staphylococcus saprophyticus, Streptobacillus moniliformis, Streptococcus, preferably S. faecalis, S. faecium and S. durans, Streptococcus agalactiae, Streptococcus pneumoniae, Streptococcus pyogenes, Treponema carateum, Treponeam pallidum, Treponema pertenue, preferably T. pallidum, Ureaplasma urealyticum, Vibrio cholerae, Vibrio parahaemolyticus, Yersinia enterocolitica, and Yersinia pestis.

In one preferred embodiment the bacterial species may be resistant to at least one antibiotic medicament. For example the bacterial species may be multiresistant.

The invention also concerns parasites, which may infect an individual. The infection by a parasite is cured by the innate immune system, which may have a decreased defence capacity in an individual having the described mutation in the MASP-2 gene. Accordingly, a parasite infection is also under scope of the present invention. Infectious parasites may for example be selected from the group consisting of Malaria (Plasmodium. falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) or Coccidiosis (Eimeria species).

Infection by fungi may also lead to development a severe disease in an individual having an impaired activity of the MBL-complement pathway due to the mutation described above. Therefore fungal infections are also covered by the present invention. A fungal infection, which may be treated by the above gene therapy vector(s) may for example be selected from the group consisting of Cryptococcus neoformans, Candida albicans, Apergillus fumigatus and Coccidioidomycosis.

In another embodiment of the present invention relates to a viral infection, that is infection by a virus.

Viruses according to the present invention may for example be selected from the group consisting of: Adeno-associated virus, Adenovirus, Avian infectious bronchitis virus, Baculovirus, Chicken pox, Corona virus, Cytomegalovirus, Distemper, Entero-virus, Epstein Barr virus, Feline leukemia virus, Flavivirus, Foot and mouth disease virus, Hepatitis A, Hepatitis B, Hepatitis C, Hepatitis E, Herpes species, Herpes simplex, Influenza virus, HIV-1, HIV-2, HTLV 1, Influenza A and B, Kunjin virus, Lassa fever virus, LCMV (lymphocytic choriomeningitis virus), lentivirus, Measles, Mengo virus, Morbillivirus, Myxovirus, Papilloma virus, Parovirus, Parainfluenza virus, Paramyxovirus, Parvovirus, Poko virus, Polio virus, Polyoma tumour virus, pseudorabies, Rabies virus, Reovirus, Respiratory syncytial virus, retrovirus, rhinovirus, Rinderpest, Rotavirus, Semliki forest virus, Sendai virus, Simian Virus 40, Sindbis virus, SV5, Tick borne encephalitis virus, Togavirus (rubella, yellow fever, dengue fever), Vaccinia virus, Venezuelan equine encephalomyelitis and Vesicular stomatis virus.

In one preferred embodiment the virus is a retrovirus, such as for example HIV (Human Immunodeficiency Virus).

In a separate aspect the invention relates to a vector comprising the nucleic acid sequence of SEQ ID NO: 3 with the G allele in position 359 operably linked to a promoter sequence capable of directing the expression of mutant MASP-2 encoded by SEQ ID NO: 3. In one embodiment this vector can be used as an expression vector in screens directed at developing drugs against immuno-related diseases.

Preferably the vector is to be used for the treating an individual having undesirably high activity of the MBL-pathway.

According to some embodiments the vector may comprise a constitutive promoter. According to other embodiments the vector may comprise a promoter sequence comprising a regulatory sequence such as a viral promoter sequence.

Medical Treatment

The MBL-pathway is a very powerful activator of the complement system. It may also be that inexpedient activation through microbial carbohydrates or endotoxins can lead to damaging inflammatory responses. The inflammatory response may be chronic, such as e.g. rheumatoid arthritis or systemic lupus erythermatosis. Compounds of the invention, such as the described above polynucleotides, polypeptides, peptide fragments and antibodies can be used as medicaments for the treatment of such conditions. The invention features these compounds as specific inhibitors of the MBL-complement pathway and they are therefore aimed for the manufacture of a medicament for treatment of conditions associated with undesirably high activity of said pathway.

The medicament comprising polynucleotides, polypeptides, peptide fragments and/or antibodies of the invention may also be used for the treatment of severe infections. In particular the medicament may be used in treatment of meningitis.

Besides the mentioned chronic inflammatory responses a medicament comprising the compounds of the invention may be directed for the treatment of reoxigenated ischemic tissues, or such the inflammatory condition that result from autoimmune condition after an acute myocardial infraction or brain ischemia.

Any pathologic condition accompanied by a massive cell loss due to apoptosis may also be considered a the condition for treatment by a medicament comprising polynucleotides, polypeptides, peptide fragments and/or antibodies of the invention.

Preferred compounds for manufacture of a medicament of the invention are
a polypeptide comprising any of the amino acid sequences set forth in SEQ ID NOS: 1 or 2, or fragments thereof, said polypeptide or said fragments comprising Gly in position 105 of said sequences;
an isolated peptide fragment having a size in a range from 5 to 160 amino acids derived from the amino acid sequence set forth in SEQ ID NO: 1 comprising at least 5 amino acid contiguous sequence, said sequence corresponding to amino acid residues 100-105, 101-106, 102-107, 103-108, 104-109 and/or 105-110 of the sequence set forth in SEQ ID NO: 1;
an isolated peptide fragment having a size in a range from 5 to 160 amino acids derived from the amino acid sequence set forth in SEQ ID NO: 1 comprising at least 5 amino acid contiguous sequence, said sequence corresponding to amino acid residues 100-105, 101-106, 102-107, 103-108, 104-109 and/or 105-110 of the sequence set forth in SEQ ID NO: 1, wherein Gly in position 105 of said sequence is substituted for Asp;
a polynucleotide encoding the above polypeptides or the peptide fragment,
a MASP-2 polypeptide having the sequence identified in SEQ ID NO: 1, variants or fragments thereof;
a MAp-19 polypeptide having the sequence identified in SEQ ID NO: 2, variants or fragments thereof;
an antibody capable of recognising an epitope comprising Asp corresponding to position 105 of the sequence identified in SEQ ID NO: 1 or 2;
an antibody capable of recognition an epitope in mutated MASP-2 and/or MAp-19, said epitope being absent in non-mutated MASP-2 and/or MAp-19, said mutated MASP-2 and/or MAp-19 having the mutation Asp105Gly.

The polypeptides comprising the sequence of MASP-2 or MAp-19, or variants or fragments thereof, wherein Asp in position corresponding to position 105 of the sequences set forth in SEQ ID NO: 1 or 2 is non-mutated, are meant by the invention for the use in treatment of pathologic conditions in an individual having the above described mutation.

The antibody capable of recognising an epitope comprising Asp corresponding to position 105 of the sequence identified in SEQ ID NO: 1 or 2, or fragments thereof, may be used as an inhibitor of binding MASP-2 to MBL, and therefore used for the treatment of conditions when a high activity of MBL-pathway is not desiarable.

According to the present invention the above compounds are used for the preparation of a pharmaceutical composition for the described above medical treatment. The pharmaceutical compositions according to the present invention may comprise one or more polypeptides or other compounds according to this invention, optionally further comprising pharmaceutically acceptable carriers.

The compositions can be administered by injection by gradual infusion over time or by any other medically acceptable mode. The administration may, for example, be intravenous, intraperitoneal, intramuscular, intracavity, subcutaneous or transdermal. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishers. electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation. When the compositions of the invention are administered for the treatment of pulmonary disorders the compositions may be delivered for example by aerosol.

The compositions of the invention are administered in therapeutically effective amounts. As used herein, an effective amount of the polypeptide or compound of the invention is a dosage, which is sufficient to conduct the desired associated complement activation or neutralization. The effective amount is sufficient to produce the desired effect of inhibiting associated cellular injury until the symptoms associated with the MBL mediated disorder are ameliorated or decreased. Preferably an effective amount of the polypeptide is an effective amount for preventing cellular injury.

Generally, a therapeutically effective amount may vary with the subject's age, condition, and sex, as well as the extent of the disease in the subject and can be determined by one of skill in the art. The dosage may be adjusted by the individual physician or veterinarian in the event of any complication. A therapeutically effective amount typically will vary from about 0.01 mg/kg to about 500 mg/kg, such as typically from about 0.1 mg/kg to about 200 mg/kg, and often from about 0.2 mg/kg to about 20 mg/kg, in one or more dose administrations daily, for one or several days (depending of course of the mode of administration and the factors discussed above).

One of skill in the art can determine what an effective amount of a compound is by screening the MASP-2 concentration and associated the MBL-complement activity in an in vitro assay.

The polypeptide and compound may be administered in a physiologically acceptable carrier. The term “physiologically-acceptable” refers to a non-toxic material that is compatible with the biological systems such of a tissue or organism. The physiologically acceptable carrier must be sterile for in vivo administration. The characteristics of the carrier will depend on the route of administration. The characteristics of the carrier will depend on the route of administration.

### EXAMPLES

The Polymorphisms of the MASP-2 protein Aps->Gly is found in 3 individuals. Below a medical case report describing the symptoms and clinical data for one of the individuals is described. The data are included with consent of the person in question.

Medical Case Report

Patient

NN, male, born in 1967, had normal childhood without notable childhood infections provided by standard vaccination programme.

At the age of 13 NN was diagnosed for ulcerative colitis. Locally installed prednisolone successfully cured the symptoms.

In 1992 NN was treated for anal condylomas.

In 1996 he developed erythema multiform bullosum with a typical clinical appearance and verified by cutaneous biopsy. Due to a diagnosis of systemic lupus erythematosus (SLE) he was treated with prednisolone. Prednisolone was later combined with other immunosuppressive drugs: azathioprine, ciclosporine, methotrexate, methotrexate plus ciclosporine and for a period also chloroquine phosphate. During this period NN had recurrent herpes infections and recurrent severe lung infections with pneumonia that is often occurring in relation to a immunosuppressive treatment.

Progressive lung fibrosis, without vasculitis, alveolitis, or granulomas, was diagnosed in 1997. NN had lost weight, felt tired and complained for dyspnoea, arthralgia, myalgia. Analyses showed slight elevations of C-reactive protein, neutropenia, a low level of complement C3 and complement C4, increased level of immunoglobulin A, slightly decreased level of immunoglobulin M. He was P-ANCA positive with a titer 1-80 and ANA positive with no clear pattern. Normal values were found for: immunoglobulin G, erythrocytes, leukocytes, thrombocytes, differential leukocyte counts, alanine amino transferase, alkaline phosphatase, urine sediment, Coomb's test, anti-cardiolipin antibody, PR3-ANCA, MPO-ANCA and C-ANCA. NN was negative for hepatitis A, B and C, and HIV.

At the present NN is diagnosed for erythema multiform bullosum, arthralgia and myalgia, relative lymphocytopenia and has slightly elevated C-reactive protein. The immunosuppressive treatment has been limited to a low dosage of prednisolone 5-15 mg daily. Analysis of NN's blood does not reveal any abnomalities in leukocyte number or function. NN has not recently has any severe bacterial infection. In case of current infections NN is normally prescribed to an antibiotic treatment.

Patient's Family

NN's father, born 1936, was operated for colon cancer in 1985, treated for myocardial infarction in 1991, and was in 1997 diagnosed with rheumatoid arthritis. He has no record of severe infections.

NN's mother, brother, son and all are essentially healthy.

Samples

Samples were EDTA plasma collected according to the standard procedure and kept at −80° C. until use.

The samples were obtained from NN on three different occasions covering a half the year when the patient was in normal and stable condition. Samples were also obtained from the parents, both children, wife, and brother of the patient.

Other samples were from 10 unrelated SLE patients presenting anti-C1q antibody. MBL deficient serum was from healthy volunteers with less than 20 ng of MBL per ml serum.

Complement Analysis

The status of the complement system was evaluated including quantification of C1q, C1s, C4, C3, C3d, Properdin, C1-inhibitor and soluble MAC complex (C5b-C9). The functional activity of the classical and the alternative pathway of complement activation as well as of the C1-inhibitor and presence of anti-C1q antibodies were also analysed.

Initial evaluations of the immune system of the patient presented in Table 1 show no other serious abnormalities than a compromised classical complement pathway. The patient had low C1q, C4 and C3, increased C3dg and had anti-C1q antibodies.

TABLE 1
Complement profile in the patient

| | | |
|---|---|---|
| Component | Value | Reference area |

| C1q | 11%¹ | | 76-136% | |
|---|---|---|---|---|
| C4 | 0.06 | g/L | 0.12-0.33 | g/L |
| C3 | 0.36 | g/L | 0.77-1.38 | g/L |
| Properdin | 54%¹ | | 57-153% | |
| C1 inhibitor | 123%¹ | | 72-146% | |
| C1 inhibitor function | 116%¹ | | 70-130% | |
| C3dg fragments | 13 | mg/L | <5 | mg/L |
| Terminal component | 43 | U/L | <20 | U/L |
| complexes (SC5b-C9) | | | | |
| Classical pathway function² | 6%¹ | | 90-112% | |
| Alternative pathway function³ | 22%¹ | | 65-161% | |
| Anti-C1q autoantibodies⁴ | 55 | U/L | <16 | U/L |

¹Percent of normal
²Hemolysis of antibody-coated sheep erythrocytes
³Hemolysis of rabbit erythrocytes
⁴Anti-C1q autoantibodies were determined by enzyme-linked immunosorbent assay.
The antibodies were not detectable by Western blot analysis.

Control sera from ten well-characterised SLE patients containing anti-C1q antibodies was examined for a capability of the MBL-MASP complexes to activate the C4 complement. All the samples showed normal activity of the MBL-MASP complexes.

Analysis of Total Plasma Concentration of MASP-2 and MAp-19

Serum samples were incubated with ant-MAp19/MASP-2 antibody coupled to Sepharose beads. Bound material was eluted and analysed by Western blotting using anti-MASP-2/MAp19 antibody.

Analysis of the blots showed the presence of MASP-2 and MAp19 in samples of the patient serum. However, the amount of MASP-2 was estimated to be less than 10 percent of the amount present in samples of serum from six control individuals. The amount of MAp19 was about 50 percent compared to control. There was no difference in the amounts of MASP-2 and MAp19 isolated from MBL sufficient serum and from MBL deficient serum of the patient.

Analysis of Molecular Composition of MBL Complexes

Diluted plasma or serum samples were incubated overnight at 4° C. in microtiter wells coated with mannan lectin. After wash the bound material in the wells was incubated with anti-MAp19/MASP-2 antibody, anti-MASP-1 antibody or anti-MASP-2 antiserum, followed by incubation with rabbit anti-rat Ig antibody (DAKO, Glostrup, Denmark) labelled with europium according to the manufacturers procedure (Wallac Oy, Turku, Finland) or rabbit anti-mouse Ig (DAKO) similarly labelled. After incubation and wash, bound europium was detected by time-resolved fluorometry. Otherwise, plasma samples, diluted as above, were incubated overnight at 4° C. in microtiter wells coated with monoclonal anti-MBL antibody (clone 131-1, Immunolex, Copenhagen, Denmark) at 5 μg/ml PBS. The bound material was eluted in SDS-PAGE sample buffer, separated by SDS-PAGE on 4-20% gels, and blotted onto PVDF membranes. The blots were incubated with anti-MAp19/MASP-2 or anti-MASP-1 antibody and consequently developed with horse radish peroxidase (HRP)-labelled rabbit anti-rat Ig (DAKO) or HRP-labelled rabbit anti-mouse Ig (DAKO), and luminescence substrate (SuperSignal, Pierce Chemicals, Rockville, Ill.). The light emission was detected by exposure the blots to a film and was quantified by recording the emission by a digital camera (Imager1000, KODAK).

Analyses of the blots revealed the presence of MASP-1 and MASP-3, but no detectable amounts of MASP-2 or MAp19 in the MBL-complexes.

Analysis of Functional Activity of the MBL Pathway

MBL-MASP complexes were purified from normal plasma by carbohydrate affinity chromatography. MBL was separated from the MASPs by ion exchange chromatography at acetic conditions.

MBL in plasma was quantified by the time resolved immunofluorimetric assay (TRIFMA). Briefly, microtiter wells were coated with monoclonal anti-MBL, incubated with several dilutions of plasma samples, afterwards followed by development with europium-labelled monoclonal anti-MBL and the consequent measurement of europium by time resolved fluorimetry.

For estimating the capacity of the MBL-MASP complexes to activate C4, microtiter wells were coated with yeast mannan lectin. Diluted plasma samples were incubated in the wells in the presence of 1 M NaCl. After washing the wells, purified human complement C4 was added and incubated at 37° C. for 1 h. Detection of deposited C4b was done by using biotinylated anti-C4 antibody and europium-labelled streptavidin.

Analyses of concentration of MBL and functional activity of the MBL-MASP complexes showed severe deficiency in the MBL pathway activity despite of the MBL level being 0.7 μg/ml. The MBL concentration in samples obtained at three different occasions was the same. Activity of the MBL pathway, expressed in relation to the MBL level showed a value below 10 mU/μg MBL.

As a control of activity of the patient MBL, MASP from MBL deficient serum collected from healthy individuals was used. Diluted 1/100 samples the MBL deficient serum were added to the wells coated with the MBL-MASP complexes from the patient and incubated for 1 hour at 37° C. Thereafter the wells were incubated with anti-C4 antibody as above. The experiment showed full activity of the patient MBL. Restoration of the MBL pathway was also achieved by adding recombinant MASP-2 to the patient serum. Additionally, restoration of the activity of the MBL-complexes from the patient was an attempted by adding to samples exogenous MBL. The addition of MBL to MBL deficient serum was able to fully reconstitute the activation of C4. However, no reconstitution was achieved by adding exogenous MBL to the patient serum. A sugar-binding capacity of the patient MBL was estimated being not impaired.

Test for the Presence of anti-MASP-2 Auto-Antibodies

Test for the Presence of immune complexes containing MAp19 and/or MASP-2: microtiter wells, coated with streptavidin and biotin labeled anti-MAp19/MASP-2 or control biotinylated normal rat IgG, were incubated with 1/50 diluted serum samples, and the bound immune complexes were consequently detected by incubation with rabbit anti-human Ig (anti-IgG H and L chain, A0107, DAKO) followed by europium-labelled chicken anti-rabbit IgG.

Test for free antibodies: microtiter wells, coated with recombinant MAp19 or human serum albumin (control), were incubated with diluted serum and developed as above.

Test for the presence of antibodies bound to MBL-MASP complexes: the wells coated with anti-MBL were incubated with diluted serum and the bound auto-antibody were detected with rabbit anti-human Ig and europium-labelled anti-rabbit IgG.

The presence of anti-MASP-2 antibodies was also evaluated by incubating Western blots of crude MBL-MASP preparations with serum samples. Briefly, strips of the blot were cut and incubated with serum samples, washed, incubated with rabbit anti-human Ig antibody (DAKO), and developed with HRP-labelled goat anti-rabbit IgG.

No anti-MASP-2 antibodies were detected in samples by any the above means.

Sequencing the MASP-2 Gene.

DNA was isolated from EDTA blood. The exons of the gene encoding MAp19/MASP-2 as well as 1100 bp of the promoter region were amplified by PCR on genomic DNA and sequenced (LARK, Essex, UK). As the sequences in certain positions showed deviations from the published sequences we subsequently used the primer sets 5′-GCGAGTACGACTTCGTCMGG-3′ (SEQ ID NO: 5) and 5′-CTCGGCTGCATAGMGGCCTC-3′ (SEQ ID NO: 6) to amplify parts of the region encoding the first domain (CUB1) and 5′-CCAGACCTTTGGAAAGTTAGC-3′ (SEQ ID NO: 7) and 5′-GGCTCAAGTTCCAAGTATTGC-3′ (SEQ ID NO: 8) to amplify part of the region encoding the fifth domain (CCP2) of MASP-2. These PCR products were then sequenced.

The MBL gene was sequenced through PCR amplification of the individual exons. MBL genotypes were determined by polymerase chain reaction with sequence specific primers.

Sequence SEQ ID NO:1 (mature MASP-2)
TPLGPKWPEPVFGRLASPGFPGEYANDQERRWTLTAPPGYRLRLYFTHFDLEL-
SHLCEYDFVKLSSGAKVLATLCGQESTDTERAPGKDTFYSLGSSLDITFRSDYS-
NEKPFTGFEAFYAAEDIDECQVAPGEAPTCDHHCHNHLGGFYCSCRAGYVL-
HRNKRTCSALCSGQVFTQRSGELSSPEYPRPYPKLSSCTYSISLEEGFSVILD-
FVESFDVETHPETLCPYDFLKIQTDREEHGPFCGKTLPHRIETKSNTVTITFVT-
DESGDHTGWKIHYTSTAQPCPYPMAPPNGHVSPVQAKYILKDSFSIFCET-
GYELLQGHLPLKSFTAVCQKDGSWDRPMPACSIVDCGPPDDLPSGRVEYITGP-
GVTTYKAVIQYSCEETFYTMKVNDGKYVCEADGFWTSSKGEKSLPVCEPVCGL-
SARTTGGRIYGGQKAKPGDFPWQVLILGGTTAAGALLYDNWVLTAAHAVYEQKH-
DASALDIRMGTLKRLSPHYTQAWSEAVFIHEGYTHDAGFDNDIALIKLNNKVVINS-
NITPICLPRKEAESFMRTDDIGTASGWGLTQRGFLARNLMYVDIPIVDHQKC-
TAAYEKPPYPRGSVTANMLCAGLESGGKDSCRGDSGGALVFLDSETERWFVG-
GIVSWGSMNCGEAGQYGVYTKVINYIPWIENIISDF
Sequence SEQ ID NO:2 (MAp-19)
TPLGPKWPEPVFGRLASPGFPGEYANDQERRWTLTAPPGYRLRLYFTHFDLEL-
SHLCEYDFVKLSSGAKVLATLCGQESTDTERAPGKDTFYSLGSSLDITFRSDYS-
NEKPFTGFEAFYAAEDIDECQVAPGEAPTCDHHCHNHLGGFYCSCRAGYVL-
HRNKRTCSEQSL
Sequence SEQ ID NO:3 (cDNA MASP-2)
atgaggctgc tgaccctcct gggccttctg tgtggctcgg tggccaccc ccttgggccc gaagtggcct
gaacctgtgt tcgggcgcct ggcatccccc ggctttccag gggagtatgc caatgaccag gagcggcgct
ggaccctgac tgcacccccc ggctaccgcc tgcgcctcta cttcacccac ttcgacctgg agctctccca
cctctgcgag tacgacttcg tcaagctgag ctcgggggcc aaggtgctgg ccacgctgtg cgggcaggag
agcacagaca cggagcgggc ccctggcaag gacactttct actcgctggg ctccagcctg gacattacct
tccgctccga ctactccaac gagaagccgt tcacggggtt cgaggccttc tatgcagccg aggacattga
cgagtgccag gtggccccgg gagaggcgcc cacctgcgac caccactgcc acaaccacct gggcggtttc
tactgctcct gccgcgcagg ctacgtcctg caccgtaaca agcgcacctg ctcagccctg tgctccggcc
aggtcttcac ccagaggtct ggggagctca gcagccctga atacccacgg ccgtatccca aactctccag
ttgcacttac agcatcagcc tggaggaggg gttcagtgtc attctggact ttgtggagtc cttcgatgtg
gagacacacc ctgaaaccct gtgtccctac gactttctca agattcaaac agacagagaa gaacatggcc
cattctgtgg gaagacattg ccccacagga ttgaaacaaa aagcaacacg gtgaccatca cctttgtcac
agatgaatca ggagaccaca caggctggaa gatccactac acgagcacag cgcagccttg cccttatccg
atggcgccac ctaatggcca cgtttcacct gtgcaagcca aatacatcct gaaagacagc ttctccatct
tttgcgagac tggctatgag cttctgcaag gtcacttgcc cctgaaatcc tttactgcag tttgtcagaa
agatggatct tgggaccggc caatgcccgc gtgcagcatt gttgactgtg gccctcctga tgatctaccc
agtggccgag tggagtacat cacaggtcct ggagtgacca cctacaaagc tgtgattcag tacagctgtg
aagagacctt ctacacaatg aaagtgaatg atggtaaata tgtgtgtgag gctgatggat tctggacgag
ctccaaagga gaaaaatcac tcccagtctg tgagcctgtt tgtggactat cagcccgcac aacaggaggg
cgtatatatg gagggcaaaa ggcaaaacct ggtgattttc cttggcaagt cctgatatta ggtggaacca
cagcagcagg tgcactttta tatgacaact gggtcctaac agctgctcat gccgtctatg agcaaaaaca
tgatgcatcc gccctggaca ttcgaatggg caccctgaaa agactatcac ctcattatac acaagcctgg
tctgaagctg tttttataca tgaaggttat actcatgatg ctggctttga caatgacata gcactgatta
aattgaataa caaagttgta atcaatagca acatcacgcc tatttgtctg ccaagaaaag aagctgaatc
ctttatgagg acagatgaca ttggaactgc atctggatgg ggattaaccc aaaggggttt tcttgctaga
aatctaatgt atgtcgacat accgattgtt gaccatcaaa aatgtactgc tgcatatgaa aagccaccct
atccaagggg aagtgtaact gctaacatgc tttgtgctgg cttagaaagt gggggcaagg acagctgcag
aggtgacagc ggaggggcac tggtgtttct agatagtgaa acagagaggt ggtttgtggg aggaatagtg
tcctggggtt ccatgaattg tggggaagca ggtcagtatg gagtctacac aaaagttatt aactatattc
cctggatcga gaacataatt agtgattttt aa
Sequence SEQ ID NO:4 (cDNA MAp-19)
atgaggctgc tgaccctcct gggccttctg tgtggctcgg tggccacccc cttgggcccg aagtggcctg
aacctgtgtt cgggcgcctg gcatcccccg gctttccagg ggagtatgcc aatgaccagg agcggcgctg
gaccctgact gcaccccccg gctaccgcct gcgcctctac ttcacccact tcgacctgga gctctcccac
ctctgcgagt acgacttcgt caagctgagc tcgggggcca aggtgctggc cacgctgtgc gggcaggaga
gcacagacac ggagcgggcc cctggcaagg acactttcta ctcgctgggc tccagcctgg acattacctt
ccgctccgac tactccaacg agaagccgtt cacggggttc gaggccttct atgcagccga ggacattgac
gagtgccagg tggccccggg agaggcgccc acctgcgacc accactgcca caaccacctg ggcggtttct
actgctcctg ccgcgcaggc tacgtcctgc accgtaacaa gcgcacctgc tcagagcaga gcctctag

1-46. (canceled)

47. A method for determining a predisposition for a manifestation of an immune system related disease in an individual comprising, determining in a biological sample isolated from said individual the presence or absence of a polymorphism, said polymorphism being located within a fragment of MASP-2 consisting of the CUB1, EGF, CUB2, CCP1 and CCP2 domains and/or located within the amino acid sequence of MAp-19,
wherein said polymorphism is within the amino acid sequence of the MASP-2 protein as identified in SEQ ID NO: 1, or the Map-19 protein as identified in SEQ ID NO: 2, said polymorphism being a substitution, deletion and/or addition of at least one amino acid residue or
wherein said polymorphism is within the coding DNA sequence of human MASP-2 as identified in SEQ ID NO: 3 or the MAp-19 protein as identified in SEQ ID NO: 4, said polymorphism being a substitution, deletion and/or addition of at least one nucleotide within said coding sequence.

48. The method of claim 47, wherein the polymorphism is a substitution, deletion and/or addition of at least one amino acid located within a fragment of MASP-2 consisting of the CUB1, EGF, CUB2 domains.

49. The method of claim 47, wherein the polymorphism is a substitution, deletion and/or addition of at least one amino acid located within a fragment of MASP-2 consisting of the CUB1 and EGF domains.

50. The method of claim 47, wherein the polymorphism is a substitution, deletion and/or addition of at least one amino acid located within a fragment of MASP-2 and/or MAp-19 consisting of CUB1.

51. The method according to claim 47, wherein the polymorphism is a substitution, deletion and/or addition of at least one amino acid located within the range of amino acid residues from position 80 to position 120 according to the amino acid sequences set forth in SEQ ID NO: 1 or 2.

52. The method according to claim 47, wherein the polymorphism being a substitution for Asp or deletion of Asp in position 105 according to the amino acid sequences set forth in SEQ ID NO: 1 or 2.

53. The method of claim 52, the polymorphism being Asp→Gly.

54. The method according to claim 47, wherein the polymorphism is determined by isolating the MASP-2 and/or MAp-19 proteins from a biological sample collected from an individual and ascertaining the substitution/mutation in the amino acid sequence of said proteins by a method selected from the group consisting of mass-spectroscopy methods, MALDI-TOF mass-spectroscopy, protein sequencing methods and immunoassays.

55. The method according to claim 47 further comprising isolating the MBL-MASP or ficolin-MASP complexes from a biological sample collected from an individual and examining the ability of the complex to activate C4 complement.

56. The method according to claim 47 further comprising examining the protein composition of MBL or ficolin complexes in a biological sample collected from an individual.

57. The method according to claim 47, wherein the predisposition to a manifestation of an immune system related disease is determined by the absence of the MASP-2 (SEQ ID NO: 1) and/or MAp19 (SEQ ID NO: 2) proteins in the MBL or ficolin complexes.

58. The method according to claim 47, wherein the presence or absence of the polymorphism is detected by hybridising a probe to a target nucleic acid sequence comprising at least position 359 according to the SEQ ID NO: 3 or SEQ ID NO: 4 or the corresponding position of the complementary strand.

59. The method of claim 47, the polymorphism being a single nucleotide substitution/mutation A→G in position 359 corresponding to the sequence set forth in SEQ ID NO: 3.

60. The method according to claim 59, wherein the probe is bound to a detectable label.

61. The method according to claim 60, wherein the label is selected from the group consisting of fluorescent reporter groups, enzyme tags, chemiluminescent groups and radioisotopes.

62. The method according to claim 59, comprising the use of a capture probe for capturing a target nucleic acid sequence.

63. The method according to claim 59, comprising amplification of a nucleotide sequence comprising the polymorphism.

64. The method according to claim 63, wherein amplification comprises use of a primer pair comprising SEQ ID NO: 5 and 6, or SEQ ID NO: 7 and 8.

65. The method according to claim 47, wherein the presence or absence of the polymorphism is detected by using isolation of a target nucleic acid from an individual, said target nucleic acid comprising at least position 359 according to the sequence set forth in the SEQ ID NO: 3 or the corresponding position of the complementary strand, and sequencing of said isolated target nucleic acid.

66. The method according to claim 47, further comprising assessing the alleles at nucleotide no. 359 according to the sequence set forth in SEQ ID NO: 3 in a target nucleotide sequence corresponding to SEQ ID NO: 3 or the complementary strand.

67. An isolated oligonucleotide comprising at least 10 contiguous nucleotides of SEQ ID NO: 3 or the corresponding complementary strand, said isolated oligonucleotide sequence comprising the G allele in position 359 or the corresponding allele of the complementary strand.

68. The isolated oligonucleotide according to claim 67, comprising at least 15 contiguous nucleotides, more preferably at least 20 nucleotides.

69. The isolated oligonucleotide according to claim 67, wherein the oligonucleotide is a polynucleotide sequence encoding the MASP-2 polypeptide having Gly at position 105 according to the amino acid sequence set forth in SEQ ID NO: 1.

70. The isolated oligonucleotide according to claim 67, wherein the oligonucleotide is a polynucleotide sequence encoding the MAp-19 polypeptide having Gly at position 105 according to the amino acid sequence set forth in SEQ ID NO: 2.

71. The isolated oligonucleotide or polynucleotide sequence according to claim 67, wherein the nucleotides are selected from the group consisting of RNA, DNA, LNA, PNA monomers and chemically modified nucleotides capable of hybridising to a target sequence.

72. An isolated antibody capable of recognition of the MASP-2 and/or MAp-19 polypeptides, or a selective binding fragment of such an antibody, wherein (1) said polypeptides and fragments comprise Gly in position 105 according to the SEQ ID NOS: 1 or 2, and said recognition is by selectively binding to an epitope comprising said Gly, or selectively binding to an epitope created within said polypeptides or said fragments, due to mutation of Asp→Gly in position 105 according to SEQ ID NOS:1 or 2, or
(2) said polypeptides and fragments comprise Asp in position 105 according to the SEQ ID NOS: 1 or 2, and said recognition is by selectively binding to an epitope comprising said Asp, or selectively binding to an epitope created within said polypeptides or said fragments, due to Asp in position 105 according to SEQ ID NOS:1 or 2.

73. A kit for predicting an increased risk of a subject of developing an immunologic disease comprising
(1) at least one probe comprising a oligonucleotide sequence, said oligonucleotide sequence comprising at least 10 contiguous oligonucleotides of SEQ ID NO: 3 or the corresponding complementary strand, said isolated oligonucleotide sequence comprising the G allele in position 359 or the corresponding allele of the complementary strand and/or
(2) at least one probe comprising at least one antibody, or a selective binding fragment of an antibody, as defined by claim 72.

74. The kit according to claim 73, wherein the probe is linked to a detectable label.

75. The kit according to claim 73, further comprising a set of primers for amplifying a region of the human MASP-2 gene, said region comprising position 359 according to SEQ ID NO: 3 or the corresponding complementary strand.

76. A method of treatment of an individual having a predisposition to a manifestation of an immune system related disease comprising
I) identification of a mutation in the MASP-2 gene of said individual and
II) administering to said individual an effective amount of a polypeptide comprising SEQ ID NO:1 and/or polypeptide comprising SEQ ID NO:2.

77. An isolated polypeptide selected from the group consisting of
(1) an isolated polypeptide comprising the amino acid sequence set forth in SEQ ID NO: 1 or SEQ ID NO 2 or a fragment thereof, said polypeptide or said fragment comprising Gly in position 105 according to said sequence,
(2) an isolated polypeptide, wherein the polypeptide is a peptide fragment having a size in a range from 5 to 160 amino acids derived from the amino acid sequence set forth in SEQ ID NO: 1 comprising at least 5 amino acid contiguous sequence, said sequence corresponding to amino acid residues 100-105, 101-106, 102-107, 103-108, 104-109 and/or 105-110 of the sequence set forth in SEQ ID NO: 1, wherein Gly in position 105 of said sequence is substituted for Asp, and
(3) an isolated polypeptide, wherein the polypeptide is a peptide fragment having a size in a range from 5 to 160 amino acids derived from the amino acid sequence set forth in SEQ ID NO: 1 comprising at least 5 amino acid contiguous sequence, said sequence corresponding to amino acid residues 100-105, 101-106, 102-107, 103-108, 104-109 and/or 105-110 of the sequence set forth in SEQ ID NO: 1.

78. A gene therapy vector for the treating pathologic conditions associated with high or low activity of MBL-pathway,
said vector comprising the sequence identified as SEQ ID NO: 3, or a fragment thereof, operably linked to a promoter sequence capable of directing the in vivo expression of MASP-2 encoded by SEQ ID NO: 3 or
said vector comprising the nucleotide sequence identified as SEQ ID NO: 3, said sequence having substitution A→G in position 359, said sequence operably linked to a promoter sequence capable of directing the in vivo expression of MASP-2 having glycine residue in position 105 according the sequence set forth in SEQ ID NO: 1.

79. The gene therapy vector according to claim 78, for treating pathologic conditions associated with low activity of MBL-pathway in a subject carrying the G allele in the position corresponding to nucleotide position 359 of the sequence identified in SEQ ID NO: 3.

80. The gene therapy vector according to claim 78 for treating pathologic conditions associated with low activity of MBL-pathway, wherein said vector comprising the sequence identified as SEQ ID NO: 3, or a fragment thereof operably linked to a promoter sequence capable of directing the in vivo expression of MASP-2 encoded by SEQ ID NO: 3.

81. The gene therapy vector of claim 78, for treating therapeutic conditions associated with pathologically high activity of the MBL-pathway, said vector comprising the nucleotide sequence identified as SEQ ID NO: 3, except that said sequence has the substitution A→G in position 359, said sequence operably linked to a promoter sequence capable of directing the in vivo expression of MASP-2 having glycine residue in position 105 according the sequence set forth in SEQ ID NO: 1.

82. A method of treatment for inhibition of activity of the lectin-complement pathway, comprising administering to an individual an effective amount of an agent selected from the group consisting of:
(1) an isolated polypeptide comprising the amino acid sequence set forth in SEQ ID NO: 1 or SEQ ID NO 2 or a fragment thereof, said polypeptide or said fragment comprising Gly in position 105 according to said sequence,
(2) an isolated oligonucleotide comprising at least 10 contiguous nucleotides of SEQ ID NO: 3 or the corresponding complementary strand, said isolated oligonucleotide sequence comprising the G allele in position 359 or the corresponding allele of the complementary strand, and
(3) an isolated antibody capable of recognition of the MASP-2 and MAp-19 polypeptides or fragments thereof by selectively binding to an epitope comprising Asp corresponding to position 105 of the sequence set forth in SEQ ID NOS: 1 or 2.

83. The method according to claim 82, for the inhibition of activity of the lectin-complement pathway, comprising administering a polypeptide sequence as set forth in SEQ ID NOS: 1 or 2, or a fragment thereof, said polypeptide or said fragment comprising Gly in position 105 of said sequences.

84. The method according to claim 82, for inhibition of activity of the lectin-complement pathway comprising administering, an isolated oligonucleotide comprising at least 10 contiguous nucleotides of SEQ ID NO: 3 or the corresponding complementary strand, said isolated oligonucleotide sequence comprising the G allele in position 359 or the corresponding allele of the complementary strand.

85. The method according to claim 82, for the inhibition of activity of the lectin-complement pathway, comprising administering an isolated antibody capable of recognition of the MASP-2 and MAp-19 polypeptides, or a selective binding fragment thereof, by selectively binding to an epitope comprising Asp corresponding to position 105 of the sequence set forth in SEQ ID NOS: 1 or 2.

86. The method according to claim 82, for treatment of therapeutic conditions associated with pathologically high activity of the lectin-complement pathway.

87. The method according to claim 82, wherein the therapeutic conditions associated with pathologically high activity of MBL-complement pathway being an inflammatory disease, ischemia, apoptosis or atherosclerosis.


SEQUENCE LISTING (8 sequences)

SEQ ID NO: 1 - protein, 671 aa, Homo sapiens
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 2 - protein, 170 aa, Homo sapiens
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSEQSL

SEQ ID NO: 3 - DNA, 2061 bp, Artificial Sequence
  cDNA encoding human MASP-2
atgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcagccttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaa

SEQ ID NO: 4 - DNA, 558 bp, Artificial Sequence
  cDNA encoding human MAp-19
atgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagagcagagcctctag

SEQ ID NO: 5 - DNA, 21 bp, Artificial Sequence
  upper PCR primer
gcgagtacgacttcgtcaagg

SEQ ID NO: 6 - DNA, 21 bp, Artificial Sequence
  lower PCR primer
ctcggctgcatagaaggcctc

SEQ ID NO: 7 - DNA, 21 bp, Artificial Sequence
  upper PCR primer
ccagacctttggaaagttagc

SEQ ID NO: 8 - DNA, 21 bp, Artificial Sequence
  lower PCR primer
ggctcaagttccaagtattgc